(19) United States
(12) Patent Application Publication
(10) Pub. No.: US 2015/0045610 A1
(43) Pub. Date: Feb. 12, 2015

(54) METHOD OF MODIFYING SPERMATOGENESIS, SPERMIOGENESIS AND/OR FERTILITY IN MAMMALS

(71) Applicant: Monell Chemical Senses Center, Philadelphia, PA (US)

(72) Inventor: Bedrich Mosinger, Cherry Hill, NJ (US)

(21) Appl. No.: 14/391,324

(22) PCT Filed: Apr. 10, 2013
(86) PCT No.: PCT/US13/35905

§ 371 Date: Oct. 8, 2014

Related U.S. Application Data
(60) Provisional application No. 61/623,244, filed on Apr. 12, 2012.

(51) Int. Cl. A61K 31/192; A61B 17/43; A61D 19/02; A61K 45/06

(52) U.S. Cl. CPC A61K 31/192; A61K 45/06; A61B 17/43; A61D 19/02; G01N 2500/04
USPC 600/35; 435/2; 514/571; 435/29; 424/430; 424/422

(57) ABSTRACT

Methods and compositions for modifying fertility and/or spermatogenesis or spermiogenesis in a male mammalian subject involve administering to the subject or contacting the subject's testis cells, germ cells or sperm with a composition that modifies the activation, nucleic acid expression, protein expression, signaling or activity of one or more T1R3 receptors or an intermediate in the signaling pathways thereof in the subject's testis cells or germ cells. In another embodiment, the method further involves administering to the subject or contacting the subject's testis cells, germ cells or sperm with a composition that modifies the activation, nucleic acid expression, protein expression, signaling or activity of G-α-gustducin, or an intermediate in the signaling pathways thereof. Methods for screening a test molecule for its effect on spermatogenesis or spermiogenesis or fertility and compositions for modifying spermatogenesis or spermiogenesis or fertility in a mammalian subject, e.g., contraceptive products, are provided.

### STATEMENT REGARDING FEDERALLY SPONSORED RESEARCH OR DEVELOPMENT

This invention was made with government support under Grant Nos. DC003155 and DK 081421 awarded by the National Institutes of Health. The government has certain rights in this invention.

### BACKGROUND OF THE INVENTION

Approximately 15% of human couples are infertile, of which male infertility accounts for 40-50%, and an estimated 7% of men experience problems in conceiving a child because of sperm defects. Overall fertility is decreasing worldwide, with the greatest effects in Western countries. In most cases the causes are unknown, although environmental factors are suspected. In almost half of infertile men, oligozoospermia or azoospermia is present despite unimpaired reproductive hormone secretion and a lack of other known determinants of infertility. Assisted conception including in vitro fertilization (IVF) and intra-cytoplasmic sperm injection is expensive, invasive and unreliable, and may cause epigenetic changes. On the other hand, the need to develop safe, simple, effective and reversible contraceptives is overwhelming.

Mammalian spermatogenesis and sperm maturation include at least three phases: 1) mitotic: stem cells in the testis differentiate into spermatogonia, which undergo a limited number of mitoses; 2) meiotic: diploid spermatogonia enter meiosis, each of them giving rise to four haploid spermatocytes; and 3) postmeiotic: haploid spermatocytes undergo the most dramatic morphological changes and transform into immature spermatozoa, which are released into the lumen of seminiferous tubules of the testis. Sperm then further mature in the epididymis and fuse with prostasomes derived from the prostate glands before being ejaculated. In the female reproductive tract, the sperm are capacitated and hyperactivated, and finally one of them initiates the acrosome reaction and fuses with the egg.

Each phase of mammalian spermatogenesis or spermiogenesis consists of many critical cellular and molecular steps, many of which are susceptible to the influence of intrinsic and extrinsic factors. For example, intrinsically, the testis expresses tissue specific or cell differentiation stage-specific genes or splicing variants that are found only in spermatogonia and spermatocytes and stored in spermatids. Mutations in at least 200 genes can adversely affect mammalian sperm production and function. Extrinsically, environmental agents such as pesticides, phytoestrogen, heavy metals, and other toxic compounds can significantly contribute to the development of infertility and subfertility, and possibly also to epigenetic changes.

Currently, the molecular mechanisms underlying spermatogenesis or spermiogenesis, sperm maturation and fertilization are not fully understood. Therefore, the treatment for infertility or subfertility is based on incomplete knowledge.

There remains a need in the art for methods and compositions for modifying fertility and spermatogenesis or spermiogenesis.

### SUMMARY OF THE INVENTION

The present disclosure relates to materials and methods for modulating the expression, activation, signaling or activity of human type I taste receptor subunit 3 (T1R3), Gα gustducin and/or their signaling pathways in testis, resulting in an effect on spermatogenesis or spermiogenesis that can be employed in methods and compositions designed for contraception and enhancing fertility.

In one aspect, a method of modifying spermatogenesis or spermiogenesis in a male mammalian subject comprises administering to the subject in need thereof a composition that modifies the activation, nucleic acid expression, protein expression, signaling or activity of one or more T1R3 receptors or an intermediate in the signaling pathways thereof in the subject's testis cells or germ cells. In one embodiment, this method includes administering to the subject in need thereof a composition that modifies the activation, nucleic acid expression, protein expression, signaling or activity of G-α-gustducin, or an intermediate in the signaling pathways thereof in the subject's testis cells or germ cells. Such modification alters the quality of sperm produced by the subject, the quantity of sperm produced by the subject, or a combination thereof.

In still another aspect, the method includes administering to the subject a composition that modifies the activation, nucleic acid expression, protein expression, signaling or activity of cyclic adenosine monophosphate (cAMP) or a downstream intermediate in the signaling pathway of cAMP.

In another aspect, the method comprises the administration of composition (a) which decreases, inhibits, blocks or down-regulates the activation, expression, signaling or activity of the T1R3 receptor or the intermediate; and the administration of composition (b) which decreases, inhibits, blocks or down-regulates the activation, expression, signaling or activity of G-α-gustducin, or the intermediate. According to this aspect, the method has a contraceptive effect by decreasing at least one of the quality or quantity or viability of sperm produced by the subject. In another embodiment of this contraceptive method, administration of composition (c) increases, up regulates or activates the activation, expression, signaling or activity of cAMP.

In another aspect, the method comprises the administration of composition (a) which provides, increases or up-regulates the activation, expression, signaling or activity of one or more T1R3 the T1R3 receptor or the intermediate; and the administration of composition (b) which provides, increases or up-regulates the activation, expression, signaling or activity of G-α-gustducin, or the intermediate. According to this embodiment, the method enhances fertility of the subject by enhancing the quality or quantity or viability of sperm produced by the subject. In another embodiment, the method entails administration of composition (c) which decreases, down regulates, inhibits or blocks the activation, expression, signaling or activity of cAMP.

In another aspect, a method for screening a test molecule for its effect on fertility or spermatogenesis or spermiogenesis comprises contacting a mammalian sperm cell, testis cell or cell line expressing a T1R3 receptor and Gα-gustducin ex vivo with a test molecule; and assaying the contacted cells or cell lines for a change in a physical or functional characteristic of the contacted cell or cell line in comparison with a reference cell or cell line contacted with a control molecule. A change in the physical or functional characteristic of the test molecule contacted cells or cell lines vs. the reference indicates a modifying effect of the test molecule on the quality, quantity or viability of the sperm. In one embodiment, the assaying step comprises assaying the contacted cell or cell line for a change indicative of T1R3 receptor activation, expression, signaling or activity and Gα-gustducin activation, expression, signaling or activity in comparison with a reference cell or cell line contacted with a control molecule. A change in a characteristic or activity of the test cell or cell line in comparison to a reference indicates a modifying effect if the test molecule on the quality, quantity or viability of sperm. In one embodiment, the assay detects a change in the cytosolic calcium concentration indicative of T1R3 receptor activation in comparison with a reference cell or cell line contacted with a control molecule. An increase or decrease in cytosolic calcium concentration of the test cells or cell lines vs. the reference indicates a modifying effect of the test molecule on the quality and/or quantity of sperm. In one embodiment, the method is a high-throughput method comprising multiple cells, cell lines, test molecules and references. In another embodiment, the assaying comprises an imaging assay or other known assay technique.

In still another aspect, a contraceptive composition for decreasing fertility in a mammalian subject comprises a ligand and/or a modifier (hereafter “ligand/modifier) or reagent that decreases, inhibits, or down-regulates the activation, expression, or activity of one or more T1R3 receptors or an intermediate in the signaling pathways thereof in the subject's testis cells, germ cells or sperm; and/or a ligand/modifier or reagent that decreases, inhibits, or down-regulates the activation, expression, or activity of G-α-gustducin, or an intermediate in the signaling pathways thereof in the subject's testis cells, germ cells or sperm. In another embodiment, this composition includes a reagent that increases, activates, or up-regulates the activation, expression, signaling, or activity of cAMP.

In another aspect, a contraceptive product comprises the above-defined compositions present in or coated onto a condom or other contraceptive implant or device.

In yet a further aspect, a composition for increasing or enhancing fertility in a mammalian subject comprises a ligand/modifier or reagent that increases, activates, or up-regulates the activation, signaling, expression, or activity of one or more T1R3 receptors or an intermediate in the signaling pathways thereof in the subject's testis cells, germ cells or sperm; and/or a ligand/modifier or reagent that increases, activates, or up-regulates the activation, expression, signaling or activity of Gα-gustducin, or an intermediate in the signaling pathways thereof in the subject's testis cells, germ cells or sperm. In certain embodiments, the composition contains a reagent that decreases, inhibits, or down-regulates the activation, expression, signaling or activity of cAMP.

In yet a further aspect, a composition for modifying spermatogenesis or spermiogenesis is designed specifically for a selected mammalian species.

In yet another aspect, the use of a composition that modifies the activation, nucleic acid expression, protein expression, signaling or activity of one or more T1R3 receptors or an intermediate in the signaling pathways thereof in the subject's testis cells or germ cells and/or modifies the activation, nucleic acid expression, protein expression, signaling or activity of Gα-gustducin, or an intermediate in the signaling pathways thereof in the subject's testis cells or germ cells for the modification of spermatogenesis or spermiogenesis is provided. Such modification alters the quality of spermatogenesis or spermiogenesis can be employed in methods and compositions designed for contraception and enhancing fertility.

Other aspects and advantages of the invention will be readily apparent from the following detailed description of the invention.

### BRIEF DESCRIPTION OF THE DRAWINGS

FIG. 1 is a bar graph showing human (h), mouse (m) and chimeric mouse/human (m/h) sweet receptors activated with 2.5 mM sucralose and assayed for inhibition by certain T1R3 inhibitors or controls, measured by DF/F in %.

FIG. 2A is a pair of photomicrographs showing GFP fluorescence in germ cells in testicular tubules from wildtype mouse (left) and showing expression of T1R3 and gustducin in testis of transgenic mice (right). Germ cells in testicular tubules of FIG. 2C are overlayed with GFP fluorescence following immunostaining.

FIG. 2B is a pair of photomicrographs showing GFP fluorescence in germ cells in testicular tubules from Line 7c mouse that expresses the chimeric mouse/human T1R3 but not endogenous T1R3 or α-gustducin (left) and showing results of in situ hybridization with T1R3 antisense probes on sections of tubules of wildtype males (right).

FIG. 2C is a pair of photomicrographs showing expression of T1R3 and gustducin in testis of transgenic mice. Germ cells in testicular tubules are immunostained with anti-α-gustducin antibody (left) and showing expression of CREM in testicular tubules from Blendy, J A et al, 1996 Nature, 380(6570):162-165 for comparison (right).

FIG. 3A is a section of PAS-stained testicular tubules of a WT male after 1 month on the clofibrate diet.

FIG. 3B is a section of PAS-stained testicular tubules of a line7c male after 1 month on the clofibrate diet. Note prominent and numerous PAS-positive giant cells in testis.

FIG. 3C is an image of sections of PAS-stained testicular tubules from a CREM null male, published in Blendy, cited above, shown for comparison.

FIG. 3D is a section of PAS stained testicular tubules of a T1R3/α-gustducin double null male on a normal diet. Note cellular debris in double-null males.

FIG. 3E is a section of PAS stained testicular tubules of a T1R3/α-gustducin double null male on a normal diet. Note cellular debris in double-null males.

FIG. 3F is a section of PAS stained epidydimi of line7c male after 1 month on the clofibrate diet. Note PAS-positive material with numerous immature cells and few spermatozoa in epididymis of line 7c males

FIG. 3G is an image of section of PAS-stained epidydimi from a T1R3/α-gustducin double null male on a normal diet. Note cellular debris in double-null males.

FIG. 3H is an image of sections of PAS-stained epidydimi from a CREM null male, published in Blendy, cited above, shown for comparison.

### DETAILED DESCRIPTION OF THE INVENTION

The methods and compositions described herein are based upon the inventors' discovery that the expression and signaling of sweet and umami (amino acid) sensing receptors (T1R3) and the G protein gustducin in male germ cells are important for spermatid development and that manipulation of their expression, activity or signaling can be employed to modify spermatogenesis, spermiogenesis or fertility in males

Thus, this invention is directed, in one embodiment, to a non-hormonal contraception method and contraceptive compositions based on specifically blocking male reproductive capability by inhibiting or blocking the signaling and expression of the male germ cell-expressed “sweet” receptor T1R3 and its G-protein Gα-gustducin. In one embodiment, both T1R3 and gustducin are inactivated or inhibited in expression and/or signaling. In another embodiment, the invention is directed to a method of enhancing fertility or improving production or quality of sperm by a male mammal by simultaneously activating, or increasing expression or signaling of the male germ cell-expressed T1R3 and Gα-gustducin.

As discussed in detail in the examples below, the inventor has found that the two molecules, T1R3 and α-gustducin, are necessary signaling molecules for male reproductive cells; and that their absence or inhibition leads to male sterility due to profound defects in spermatogenesis or spermiogenesis Inhibitors of GPCRs and their signaling pathways are among the most common types of medicinal drugs. Importantly, widely used phenoxy auxin herbicides and pharmaceuticals known as fibrates, are potent T1R3 inhibitors and are human specific (and thus would not be detected in rodent assays). Based on the data in the examples below, these chemicals, alone or in combination with chemicals that act as inhibitors of Gα-gustducin (or downstream phosphodiesterases), therefore affect growth or function of human male germ cells and may be partly responsible for some forms of human male infertility in genetically susceptible individuals, whose etiologies were previously unrecognized.

Combinatorial use of drugs and environmental agents, such as phenoxy-auxin herbicides, that act on T1R3, α-gustducin, or their signaling pathways result in infertility or sterility. Likewise, the combination of drugs acting on one or more of these targets can result in infertility. According to one aspect of this invention one or a combination of local or tissue-specific inhibitors of these molecules serves as an effective and reversible male nonhormonal contraceptive. According to another aspect of this invention, methods to improve spermatogenesis or spermiogenesis in men that are hypofertile due to dysfunctional cAMP/CREM signaling involve activating these molecules or their signaling pathways.

Without wishing to be bound by theory, the inventor proposes that absence or inhibition of the G-protein coupled receptor (GPCR) T1R3 and the G-protein subunit, Gα-gustducin, affects function of CREM (cAMP-responsive element modulator), the major transcription factor in developing testicular cells. This causes abnormal cAMP levels in spermatocytes and spermatids, resulting in reproductive deficiency, e.g., a block of spermatogenesis or spermiogenesis. The inventors found that Gα-gustducin, T1R3, and CREM are all expressed in the same testicular cells.

Inhibition of T1R3 and Gα-gustducin or their signaling pathways are theorized to prevent the proper splice forms of CREM from being produced and/or to cause the forms of CREM produced to be not properly functional. Alternatively, an increased protein kinase A (PKA) activity may phosphorylate CREM (normally not phosphorylated in spermatocytes) which then can associate with CREB binding protein (CBP) and start transcription of genes that are not specific to developing spermatocytes, resulting in products that are not needed or products with functions deleterious or opposite to those specified by the unphosphorylated CREM+ACT factors. This effect results in a repression in spermatogenesis or spermiogenesis and may alter sperm production, maturation, sperm motility, fertilization ability, and/or cellular functions such as morphological change, DNA or histone modification and chromatin packaging, leading to the reduced sperm function or epigenetic alterations.

If both T1R3 and α-gustducin are absent from cells that normally express these proteins, there is a likely disruption in cAMP signaling, which in turn would affect downstream cAMP-responsive molecules such as CREM. In round spermatids, an impairment of cAMP/CREM results in a severe loss of function. As shown below, the absence of T1R3 and α-gustducin genes results in transmission ratio distortion (TRD), and interferes with growth or function of male haploid germ cells. The examples below present data that show that a block of the T1R3 receptor in the background of α-gustducin-null mice results in azoospermia.

Inhibition or activation of the T1R3 and/or α-gustducin and/or cAMP and their respective signaling pathways by appropriate ligand/modifier(s) thus permit the manipulation and modification of certain characteristics of spermatogenesis or spermiogenesis and production of viable sperm, e.g., the quality and quantity of sperm as well as affect the fertilization process. As supported by the data of the examples, the inventors have identified new infertility and subfertility treatments and contraceptive measures, and compositions for use in humans, as well as in animal breeding and pest animal control. These methods and compositions also provide assays for testing sweet and unami taste sensitivity of animals or humans with polymorphisms alternative to expensive oral testing or cumbersome heterologous expression. Compounds that have an effect on male fertility could be identified by their effect on taste. If such compounds are then absorbed in the body, and remain functionally unchanged in blood (and pass the weak blood testis barrier), then they have an effect on spermatogenesis or spermiogenesis.

### I. COMPONENTS OF THE METHODS AND COMPOSITIONS

All scientific and technical terms used herein have their known and normal meaning to a person of skill in the fields of biology, biotechnology and molecular biology and by reference to published texts, which provide one skilled in the art with a general guide to many of the terms used in the present application. However, for clarity, the following terms are defined as follows:

By “mammalian subject” is meant primarily a human, but also domestic animals, e.g., dogs, cats; and livestock, such as cattle, pigs, etc.; and common laboratory mammals, such as primates, rabbits, and rodents; and pest or wild animals, such as deer, rodents, rabbits, squirrels, etc.

“T1R3” (alternative name TAS1R3) is a G-protein-coupled receptor (GPCR) and a key component of the sweet and umami (amino-acid) heterodimeric receptors expressed primarily in taste cells and enteroendocrine cells of the gastrointestinal tract, and also in the pancreatic islets and in brain. T1R3 senses sugars and promotes hormone release to regulate insulin and glucose levels. T1R3 is also expressed in testes.

“Gustducin”, as used herein, generally refers to the α-subunit of the heterotrimeric G protein which in taste cells, is formed by the association among Gα-gustducin (a Gα subunit), with a Gβ1 subunit and a Gγ13 subunit. It is also expressed in a number of extra-oral tissues, such as in the enteroendocrine cells of the gastrointestinal tract, pancreas and testis. Gα-gustducin is the major cellular downstream signaling element that couples with T1R3 and propagates the signal from activated receptor inside the cells.

By “PDE” is meant a cyclic nucleotide phosphodiesterase (PDE), which describe a group of enzymes that degrade the phosphodiester bond in the second messenger molecules cAMP and cGMP. PDEs regulate the localization, duration, and amplitude of cyclic nucleotide signaling within subcellular domains. PDEs are therefore important regulators of signal transduction mediated by these second messenger molecules. They are widely expressed; however, many have tissue-specific distribution. PDE1, PDE4, and PDE11 are expressed strongly in testis.

By “CREM” is meant the cyclic adenosine monophosphate (cAMP)—responsive element modulator, which is a protein that is encoded by the CREM gene. This gene encodes a bZIP transcription factor that binds to the cAMP responsive element found in many viral and cellular promoters. It is an important component of cAMP-mediated signal transduction during the spermatogenetic cycle, as well as other complex processes. Multiple alternatively spliced transcript variants encoding several different isoforms have been found for this gene, with some of them functioning as activators and some as repressors of transcription. CREM, in round spermatids, is indispensable for initiation of transcription of genes required for maturation of spermatocytes into functional sperm cells. CREM gene disruption results in specific male sterility.

By “CREM KO”, alternatively CREM null and CREM−/−, is a breed of knockout mice that lack the CREM gene. In these mutant animals females are fertile, whereas males are sterile due to a complete block in spermatogenesis or spermiogenesis.

By “PKA” is meant protein kinase A (PKA), which refers to a family of enzymes whose activity is dependent on cellular levels of cyclic AMP (cAMP). PKA is also known as cAMP-dependent protein kinase. PKA has several functions in the cell, including regulation of glycogen, sugar, and lipid metabolism. PKA is known to phosphorylate CREB (structurally related to CREM, but much more widely expressed transcriptional factor). PKA also can phosphorylate CREM. Under physiological conditions, CREM is not phosphorylated in developing spermatocytes in testes. Unphosphorylated CREM in cooperation with ACT transcription factor starts transcription of novel genes and thus products that are indispensable for development of unique features of sperm cells.

The term “homolog” or “homologous” as used herein with respect to any human target sequence (e.g., T1R3 or gustducin or cAMP, etc) means a nucleic acid sequence or amino acid sequence having at least 35% identity with the mRNA or protein sequence, respectively, of the target sequence, e.g., of a specific human T1R3 receptor, used for comparison and encoding a gene or protein having substantially similar function to that of the reference sequence. Such homologous sequences can be orthologs, e.g., genes in different species derived from a common ancestor. In other embodiments, the homolog can have at least 40, 50, 60%, 70%, 80%, 90% or at least 99% identity with the respective human target sequence. In one embodiment, the homolog is that of a non-human mammalian species, e.g., such as the murine T1R3 receptors identified in the examples below. Based on the known and publically available sequences of these receptors and the available computer programs readily available, such as the BLAST program, one of skill in the art can readily obtain full-length homologs, orthologs or suitable fragments of the target genes or proteins referred to herein from a mammalian species.

By “T1R3 ligand/modifier(s)” as used herein is meant a compound, molecule (synthetic or natural) or a composition comprising multiple compounds or molecules) that modify the activation, nucleic acid expression, protein expression, signaling or activity of one or more T1R3 receptors or an intermediate in the signaling pathways thereof. In one embodiment the ligand/modifier(s) operates in all cells in which T1R3 is present. In another embodiment, the ligand/modifier(s) is specific for modifying T1R3 in the subject's testis cells or germ cells or sperm. In one embodiment, the T1R3 ligand/modifier(s) decreases, inhibits, blocks or down-regulates the activation, expression, signaling or activity of a T1R3 receptor or an intermediate in the signaling pathways thereof. In another embodiment, the T1R3 ligand/modifier(s) provides, increases or up-regulates the activation, expression, signaling or activity of aT1R3 receptor or an intermediate in the signaling pathways thereof. In another embodiment, the ligand/modifier(s) exerts in modifying action on a single T1R3 receptor or intermediate or on multiple T1R3 receptors or intermediates.

Among such T1R3 ligand/modifier(s) are widely used phenoxy-auxin herbicides and structurally related anti-hyperlipidemia fibrate drugs, such as 2,4-DP, 2,4,5-TPP, MCPP, 2,4-D, clofibric acid, gemfibrozil, bezafibric acid, gymnemic acid, an inhibitor of gustducin, an inhibitor of the G α subunit of gustducin, an inhibitor of protein lipase C (PLC) beta and an inhibitor of protein kinase A. Among such ligand/modifier(s) are well-known phenoxy compounds and others disclosed in the inventors co-pending US published patent application US2010/051135 and in international patent application No. WO2011/041686, and in Maillet et al. 2009 J. Med. Chem., 52(21):6931-6935, all incorporated by reference herein. As discussed below, some of these ligand/modifier(s) act at micromolar concentrations to specifically inhibit the human sweet and umami receptors, acting upon the T1R3 subunit. Rodent T1R3 is insensitive to these compounds so that actions on these receptors would not have shown up in animal models or rodent derived cell lines. Known human-specific T1R3 inhibitors include, without limitation, antilipid fibrate drugs and phenoxy compounds such as lactisole, clofibric acid and 2-(2,4-dichlorophenoxy) propionic acid herbicide. Fibrates block T1R3 with the same potency as they activate PPARα, suggesting that this may be an important target of their action. Clofibric and bezafibric acids also inhibit the T1R3 receptors at efficiencies comparable to their activation of PPAR-α (Wilson et al, 2000 J. Med. Chem., 43:527-550). Such compounds 10× more effective against the T1R3 than clofibric acid are known (Maillet, cited above). Such antilipid drugs are known to reach systemic levels in vivo that suffice to block T1R3 receptors and have no activity on rodent receptors.

Other T1R3 ligand/modifier(s) are naturally occurring and man-made sweet substances (sugars and artificial sweeteners), which are known ligand/modifier(s) of the sweet T1R2+T1R3 receptor (Max, M., and Meyerhof, W. 2008. Taste Receptors. In The Senses: A Comprehensive Reference (Ed R. R. Hoy, et al.; Zhao G. Q., et al., 2003 Cell, 115: 255-266; Kinnamon S C, 2009 Ann N Y Acad Sci., 1170:55-9). Similarly, amino acids such as monosodium glutamate (MSG) are ligand/modifier(s) of the amino acid (umami) T1R1+T1R3 receptor (Staels, B., and Fruchart, J. C. 2005 Diabetes 54, 2460-2470.). A number of naturally occurring anti-sweet or sweet-modifying substances are suspected or proven ligand/modifier(s) of the sweet receptor (Jiang P, et al., 2005 J Biol Chem., 280(15):15238-46; Kanetkar, P., et al., 2007 J. Clin. Biochem. Nutri. 41, 77-81).

Still other T1R3 ligand/modifier(s) are phenoxy auxin herbicides, synthetic herbicides that mimic the action of auxin plant hormones (Maillet, cited above). These herbicides are extensively used (Vainio et al, 1983 Biochem Pharmacol, 32:2775-2669) in crop agriculture and in landscape turf management (Szmedra et al, 1997 Weed Science 1997:45:592-598). The IC₅₀ for T1R3 of 2,4-DP is about 5 μM (Maillet, cited above). Indeed, this is the most potent inhibitor of the human T1R3 receptor known. Because they are specific to the human form of T1R3, their effects on male reproduction and endocrine functions would have gone undetected in studies on rodents.

Certain embodiments of the invention employ ligand/modifier(s) that are T1R3 activators, such as sodium cyclamate, neohesperidine dihydrochalcone and related dihydrochalcones from plants: e.g. trilobatin, sieboldin, vacciniifolin, and confuside. Still other activators or T1R3 or intermediates in its signaling pathway, e.g., indirect T1R3 activators, include known amino acids acting on T1R1/T1R3 dimers, sugars and artificial sweeteners acting on T1R2/T1R3 dimers, D-amino acids acting on T1R2/T1R3 dimers. Suitable activators may include the sweet and umami taste enhancers acting on T1R1+3 or T1R2+3 heterodimers descried by Senomix, Inc. Other activators include sodium cyclamate, dihydrochalcon-related sweeteners, a peptide or protein or a D-amino acid acting on T1R1/T1R3 dimers, a sugar, an artificial sweetener, sucrose, maltose, fructose, a sweet alcohol, sorbitol, xylitol, erythritol, aspartame, sucralose, saccharin or monosodium glutamate and amino acids. These compounds act either in cooperative way with T1R receptors or modulate their signaling pathway (Servant G, et al., 2010 Proc Natl Acad Sci USA., 107(10):4746-51; Zhang F, et al., 2010 Proc Natl Acad Sci USA. 107(10):4752-7). Any of such molecules could potentially stimulate/activate or co-stimulate T1R3 and or gustducin pathways for use in methods and compositions for improving male fertility, particularly in cases where male fertility is due to block in these molecules' signaling. Similarly mammal-specific small molecule inhibitors or activators of T1R3 or its intermediates may be readily identified from libraries of various chemical compounds.

Still other ligand/modifier(s) of T1R3 or its intermediates can include a nucleic acid molecule that reduces or silences expression of the T1R3 receptor or its intermediates, such as through gene therapy mechanism, such as siRNA, shRNA or other known silencing reagents. In an analogous manner, other ligand/modifier(s) of the T1R3 receptor or its intermediates include a nucleic acid molecule that increases or amplifies expression of the T1R3 receptor its intermediates, via a gene therapy intervention.

By “Gustducin ligand/modifier(s)” as used herein is meant a compound, molecule (synthetic or natural) or a composition comprising multiple compounds or molecules) that modify or modulate the activation, nucleic acid expression, protein expression, signaling or activity of G-α gustducin or an intermediate in the signaling pathways thereof. Such ligand/modifier(s) can include one or more of an inhibitor of gustducin, an inhibitor of the G a subunit of gustducin, an inhibitor of protein lipase C (PLC) beta, an inhibitor of PKA, or an inhibitor of PDE. In other aspects of the compositions and methods described herein the ligand/modifier(s) have the opposing activity on gustducin. In one embodiment the ligand/modifier(s) operates in all cells in which G-α gustducin is present. In another embodiment, the ligand/modifier(s) is specific for modifying G-α gustducin in the subject's testis cells or germ cells or sperm. In one embodiment, the G-α gustducin ligand/modifier(s) decreases, inhibits, blocks or down-regulates the activation, expression, signaling or activity of a G-α gustducin receptor or an intermediate in the signaling pathways thereof. In another embodiment, the G-α gustducin ligand/modifier(s) provides, increases or up-regulates the activation, expression, signaling or activity of G-α gustducin or an intermediate in the signaling pathways thereof. In another embodiment, the ligand/modifier(s) exerts in modifying action on a single G-α gustducin or intermediate or on multiple G-α gustducin or intermediates. While there are currently no known specific known inhibitors of α-gustducin, the screening methods described herein can be used to identify same. Similarly, since Gα-gustducin activates phosphodiesterases (PDEs), a target of several prevalent medicines, Gustducin ligand/modifier(s) include those compounds, molecules or compositions that modulate or modify intermediates in its pathway, e.g., PDEs.

Gustducin ligand/modifier(s) also include several nonspecific inhibitors of Gα-proteins, such as pertussis toxin, which inhibits Gi, Go, Gα-gustducin, and Gt; and suramin and NF023, which are more selective for recombinant Gi1-α and recombinant Go-α, respectively (61-62). The G protein-coupled receptors that couple to the Gₛα subunit or Gₛ protein include without limitation, 5-HT receptors types 5-HT4 and 5-HT7, ACTH receptor, Adenosine receptor types A2a and A2b, Arginine vasopressin receptor 2, β-adrenergic receptors types β1, β2 and β3, Calcitonin receptor, Calcitonin gene-related peptide receptor, Corticotropin-releasing hormone receptor, Dopamine receptors, D1-like family (D1 and D5), FSH-receptor, Gastric inhibitory polypeptide receptor, Glucagon receptor, Histamine H2 receptor, Luteinizing hormone/choriogonadotropin receptor, Melanocortin receptor, Parathyroid hormone receptor 1, Prostaglandin receptor types D2 and I2, Secretin receptor, and Thyrotropin receptor. Certain of the T1R3 ligand/modifier(s) identified above may also act either in cooperative way with T1R receptors or modulate their signaling pathway (Servant et al, cited above; Zhang et al, cited above). Any of such molecules could potentially co-stimulate T1R3 and or gustducin pathways and improve male fertility, particularly in cases where male fertility is due to block in these molecules' signaling. Similarly mammal-specific small molecule inhibitors or activators of gustducin may be readily identified from libraries of various chemical compounds.

Still other ligand/modifier(s) of gustducin or its intermediates can include a nucleic acid molecule that reduces or silences expression of the gustducin or its intermediates, such as through gene therapy mechanism, such as siRNA, shRNA or other known silencing reagents. In an analogous manner, other ligand/modifier(s) of the gustducin or its intermediates include a nucleic acid molecule that increases or amplifies expression of the T1R3 receptor its intermediates, via a gene therapy intervention.

Because T1R3 and gustducin exert their activities in reproduction mainly through modulation of cAMP, any agent that is known to modify or modulate the activation, nucleic acid expression, protein expression, signaling or activity of cyclic adenonsine monophosphate cAMP or a downstream intermediate in the signaling pathway of cAMP can be employed as a T1R3 ligand/modifier(s) or Gustducin ligand/modifier(s) according to the methods and compositions described herein. Similarly mammal-specific small molecule inhibitors or activators of cAMP may be readily identified from libraries of various chemical compounds. For example, several bacteria produce powerful toxins that either activate adenyl cyclase or prevent its inhibition, including, without limitation, Pertussis toxin, Cholera toxin, and Anthrax toxin. Such a cAMP ligand/modifier(s) may be also e.g. a PDE ligand/modifier(s), and/or a PKA ligand/modifier(s). Such a ligand/modifier(s) may increase, up regulate or activate the activation, expression, signaling or activity of cAMP for use in methods of contraception. For this embodiment, the cAMP ligand/modifier(s) is a PDE inhibitor or a PKA activator, or an activator of adenylyl cyclase such as forskolin, an adrenaline stimulant, a synthetic analog of cAMP, 8-bromo-cAMP, dibutyryl-cAMP, or 6-benzyl-cAMP or a GPCR receptor that couples to Gₛα subunit, adrenalin, dopamine, serotonin, glucagon, or histamine. As for the T1R3 or gustducin, gene therapy silencing or amplifying agents can also be employed as appropriate ligand/modifier(s).

By “PDE ligand/modifier(s)” as used herein is meant a compound that modifies or modulates the activation, nucleic acid expression, protein expression, signaling or activity of a PDE or an intermediate in the signaling pathways thereof. In one embodiment, the ligand/modifier(s) activates PDE. In another embodiment, the ligand/modifier(s) inhibits PDE. In another embodiment, the ligand/modifier(s) activates multiple different PDE. A PDE inhibitor can be nonspecific, such as caffeine or theophylline, or specific to PDE expressed in testis, e.g. PDE1, PDE4, PDE8, PDE11 etc.

The term “test molecule” as used herein can refer to any known or novel molecule for testing as a ligand/modifier(s) that can modify or modulate the activation, nucleic acid expression, protein expression, signaling or activity of mammalian T1R3 receptor, and/or Gustducin and/or PDE for safe use in humans and or other mammals. Such molecules may typically be found in known libraries of molecules, including those that have been pre-screened e.g., for safe use in animals. Suitable test molecules may be found, for example, in AMES library and may be readily obtained from vendors such as Otava, TimTec, Inc., Chem Bridge Corp., etc. See e.g., Bhal et al, 2007 Mol. Pharmaceutics, 4(4):556-56. The test molecules/compounds identified by the methods of this invention may be chemical compounds, small molecules, nucleic acid sequences, such as cDNAs, or peptides or polypeptides, which modify or modulate, appropriately, the T1R3 receptor, α-Gustducin, cAMP, or PDE or one of the intermediates in their signal pathways in male germ cells, and affect the characteristics of spermatogenesis or spermiogenesis or the sperm produced. These test molecules may be used to modulate fertility as described herein. The compounds discussed herein also encompass “metabolites” which are unique products formed by processing the compounds of the invention by the cell or subject. Desirably, metabolites are formed in vivo.

“Male germ cell”, as used herein, refers to a sperm cell or one of its developmental precursors, including cells found in the testes of the male mammal, as well as cell lines created therefrom, spermatozoa and spermatids (collectively referred to as “sperm”).

A “transformed cell” or “cell line” as used herein refers to a mammalian cell or cell line that is genetically engineered to express a desired T1R3 mRNA or protein and/or gustducin mRNA or protein, or an intermediate thereof that it does not naturally express or that it does not naturally express it in the known amounts. Particularly desirable cells or cell lines are selected from among any mammalian species, including, without limitation, cells such as A549, WEHI, 3T3, 10T1/2, HEK 293 cells, PERC6, Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention.

By “expression level” is meant the quantitative expression of the nucleotide sequence (e.g., mRNA) of a desired target sequence or the quantitative expression of the desired expressed protein receptor itself.

The terms “control”, “reference”, “control subject” or “reference subject” are used interchangeably and refer to both an individual germ cell or germ cell line or the pooled data derived from multiple cells or cell lines or to numerical or graphical averages of the physical or functional changes in such cells or cell lines. Such controls are the types that are commonly used in diagnostic assays for other receptors, G protein subunits, cAMP or intermediates in the respective signaling pathways thereof. Selection of the particular class of controls depends upon the use to which the methods and compositions are to be put by the physician. As used herein, the term “predetermined control” refers to a numerical level, average, mean or average range of the characteristic, e.g., signaling of a T1R3 receptor in a defined male germ cell or male germ cell line population or a pattern of multiple changes for multiple physical or functional characteristics of the germ cell or cell line. The predetermined control level is preferably provided by using the same assay technique as is used for measurement of the effect of the test molecule on the cell or cell lines, to avoid any error in standardization. For example, the control may comprise a sample of sperm from one or more healthy male mammalian subjects contacted with a “control” compound, with which contact produces no physical or functional change. This reference or control can refer to a numerical average, mean or average range of the physical or functional characteristic being measured in the male germ cells or germ cell lines. It is also possible that the reference can be a sperm sample of the same subject, which is not contacted with the test molecule but with the inert control molecule.

The terms “compound”, “composition”, “reagent” or “molecule” as used herein may be used interchangeably.

The terms “a” or “an” refers to one or more, for example, “an assay” is understood to represent one or more assays. As such, the terms “a” (or “an”), “one or more,” and “at least one” are used interchangeably herein. As used herein, the term “about” means a variability of 10% from the reference given, unless otherwise specified. While various embodiments in the specification are presented using “comprising” language, under other circumstances, a related embodiment is also intended to be interpreted and described using “consisting of” or “consisting essentially of” language.

### II. METHODS

In one aspect, a method of modifying fertility and specifically spermatogenesis or spermiogenesis in a male mammalian subject comprises administering to the subject in need thereof a composition that modifies the activation, nucleic acid expression, protein expression, signaling or activity of one or more T1R3 receptors or an intermediate in the signaling pathways thereof in the subject's testis cells or germ cells. In one embodiment of this method, another step involves administering to the subject in need thereof a composition that modifies the activation, nucleic acid expression, protein expression, signaling or activity of G-α-gustducin, or an intermediate in the signaling pathways thereof in the subject's testis cells or germ cells. In certain embodiments, the intermediate in the G-α-gustducin signaling pathway is a cyclic nucleotide phosphodiesterase (PDE). In still another embodiment, the compositions that modify T1R3 and G-α-gustducin or their downstream signaling, can comprise a single compound or molecule as discussed in detail above. The modification of T1R3 and/or G-α-gustducin expression, activity or signaling alters the quality or characteristic of spermatogenesis or spermiogenesis or of sperm produced by the subject, the quantity of sperm produced by the subject, or a combination thereof.

In another embodiment, the method of modifying spermatogenesis or spermiogenesis involves administering to the subject a composition that modifies the activation, nucleic acid expression, protein expression, signaling or activity of cyclic adenosine monophosphate (cAMP) or a downstream intermediate in the signaling pathway of cAMP. In certain embodiments, the downstream intermediate in the cAMP signaling pathway is protein kinase A (PKA). The selection of the ligand/modifier(s) of T1R3, gustducin, cAMP or their respective signal pathway intermediates may be made from the specified ligand/modifier(s) defined above or from ligand/modifier(s) identified via a screening method.

The selected ligand/modifier(s) can include one or more of the ligand/modifier(s) specifically identified above. Preferably these ligand/modifier(s) are selected that have the characteristics of safe use in humans or animals. In certain embodiments, and depending upon the route of administration, a selected ligand/modifier(s) is one that can pass the blood-testis barrier. Additionally, it may be desirable to administer more than one ligand/modifier(s) in combination in a single composition, or serially or over a selected period of time.

These methods primarily involve administering the composition(s) or ligand/modifier(s) in vivo to a male subject via a suitable route. In yet another embodiment, the administration is ex vivo and involves treating a sample of a mammalian subject's germ cells or sperm with a sufficient amount of the compositions or ligand/modifier(s) that modify T1R3 and/or G-α-gustducin and/or cAMP or their respective signaling pathway intermediates.

Thus, for administration in vivo, the ligand/modifier(s) is desirably administered as a pharmaceutical composition as described below. The methods described herein involve administering the composition(s) modifying T1R3 and/ G-α-gustducin and/or cAMP or their respective signaling pathways by independent routes or the same routes of administration. Such routes are not limiting, but may include any suitable route of administration based upon the formulation of, and choice of ligand/modifier(s) in the composition. Such routes include oral, topical, intravenous injection, intraperitoneal injection, or via an implant. In certain embodiment, the route of administration comprises a sustained drug-delivery formulation to achieve steady levels of the composition(s), e.g., for contraception. In one embodiment, the ligand/modifier(s)—containing composition is administered topically, such as by a créme or ointment, or transdermal patch. In another embodiment, the ligand/modifier-containing composition is administered by intravenous injection at dosages sufficient to permit an effective amount to be delivered to the testis. In another embodiment, the ligand/modifier-containing composition is administered via an implant for timed release of suitable dosages. In this embodiment, the implant may be located close to the testis.

Depending upon the selection of the targeted T1R3 and/or G-α-gustducin and/or cAMP or their respective signaling pathway intermediates, ligand/modifier(s), route of administration, the physical characteristics of the subject, and the purpose, e.g., enhancement of fertility or contraception, a suitable dosage of the ligand/modifier(s) individually or in combination in the compositions is between 1 nM to 100 mM ligand/modifier(s) per kg body weight. In one embodiment, the dosage is at least 5 nM/kg. In another embodiment, the dosage is at least 10, 25, 50, 100, 200, 250, 300, 400, 500, 750, 800, or at least 900 nm/kg. In another embodiment, the dosage is at least 1 μM/kg. In one embodiment, the dosage is at least 5 μM/kg. In another embodiment, the dosage is at least 10, 25, 50, 100, 200, 250, 300, 400, 500, 750, 800, or at least 900 μM/kg. In another embodiment, the dosage is at least 1 mM/kg. In one embodiment, the dosage of ligand/modifier(s) is at least 5 μM/kg. In another embodiment, the dosage is at least 10 mM/kg. Dosages of each composition(s) modifying T1R3 and/ G-α-gustducin and/or cAMP or their respective signaling pathways will generally range from about 1 nM to 10 mM of active molecule or reagent or ligand/modifier(s). In another embodiment, the composition dosage of such composition(s) or a combination thereof range from about 1 μM to about 100 μM active molecule. The compositions that contain cAMP/cAMP signal pathway modifying ligand/modifier(s) or reagents may alternatively use lower dosages of the T1R3 and/ G-α-gustducin/signaling pathway ligand/modifier(s) or modifying compositions.

According to any of the methods described herein, the mammalian T1R3 receptor or an intermediate in its pathway is a human T1R3 receptor, or intermediate or a homolog or ortholog thereof from a non-human mammal. The homolog is a receptor having an amino acid sequence that is at least 95% to at least 99% homologous to a human T1R3 receptor. Wherein the intended target of the method is a non-human animal, that animals T1R3 receptor or its homolog/ortholog can be employed.

According to any of the methods described herein, the mammalian G-α-gustducin is a human G-α-gustducin, or its signal pathway intermediate, or a homolog or ortholog thereof from a non-human mammal. The homolog is a G-protein having an amino acid sequence and/or nucleic acid sequence that is at least 95% to at least 99% homologous to a human G-α-gustducin. Wherein the intended target of the method is a non-human animal, that animal's G-α-gustducin or its homolog/ortholog can be employed.

According to any of the methods described herein, the mammalian cAMP is a human cAMP, or intermediate or a homolog or ortholog thereof from a non-human mammal. The homolog is a receptor having an amino acid sequence and/or nucleic acid sequence that is at least 95% to at least 99% homologous to a human cAMP. Wherein the intended target of the method is a non-human animal, that animal's cAMP, signal pathway intermediate, or its homolog/ortholog can be employed.

As discussed above, where the subject is a human, the mammalian T1R3 receptor, and/or gustducin, and/or cAMP is a human molecule, and thus the selection of the appropriate ligand/modifier(s) or composition for administration employs a suitably species-specific molecule. Where the subject is a non-human animal, e.g., livestock for breeding, etc., the targets is appropriate homolog or ortholog from a non-human mammal and the selected ligand/modifier(s) are appropriately specific to that target.

A. Contraceptive Methods

According to a contraceptive aspect of the methods described herein, the method involves administering a composition or ligand/modifier(s) or molecule, as defined in detail above, that decreases, inhibits, blocks or down-regulates the activation, expression, signaling or activity of the T1R3 receptor or the intermediate. The method also involves in one embodiment the administration of a composition or ligand/modifier(s) or molecule, as defined in detail above, that decreases, inhibits, blocks or down-regulates the activation, expression, signaling or activity of G-α-gustducin, or the intermediate. According to another embodiment of the method, another composition, molecule or ligand/modifier(s) increases, up regulates or activates the activation, expression, signaling or activity of cAMP and is administered to the subject with the T1R3 ligand/modifier(s) and/or gustducin ligand/modifier(s). In one embodiment, the active molecule in such a composition is a PDE inhibitor. In another embodiment, the active molecule in such a composition stimulates PKA activity. Such compositions, compounds or molecules may include any of those identified in detail above which have the intended effect on the targeted T1R3 and/or gustducin and/or cAMP and/or their respective pathway intermediates. In this embodiment, administration can be direct, systemic or local to the male subject as discussed above.

Such methods have a contraceptive effect by decreasing or inhibiting spermatogenesis or spermiogenesis or inhibiting some other characteristic of spermatogenesis or spermiogenesis or sperm, e.g., the quantity or viability of sperm produced by the subject.

In still another embodiment, a method of modifying fertility in a male mammalian subject involves contacting the subject's testis cells, germ cells or sperm with a sufficient amount of a composition or ligand/modifier(s) or molecule, as defined in detail above, that decreases, inhibits, blocks or down-regulates the activation, expression, signaling or activity of the T1R3 receptor or its signal pathway intermediates expressed on or in the cells. In still another embodiment, a method of modifying fertility in a male mammalian subject involves contacting the subject's testis cells, germ cells or sperm with a sufficient amount of a composition or ligand/modifier(s) or molecule, as defined in detail above, that decreases, inhibits, blocks or down-regulates the activation, expression, signaling or activity of G-α-gustducin, or its signal pathway intermediates expressed on or in the cells. In still another embodiment, a method of modifying fertility in a male mammalian subject involves contacting the subject's testis cells, germ cells or sperm with a sufficient amount of a composition or ligand/modifier(s) or molecule, as defined in detail above, that increases, up regulates or activates the activation, expression, signaling or activity of cAMP or its signal pathway intermediates. According to this embodiment, the contacting of the selected ligand/modifier(s) and the target receptor, gustducin, cAMP or respective pathway intermediates expressed in or on the cells or sperm occurs ex vivo. This method includes treating a sample of a mammalian subject's sperm with a sufficient amount of the specified ligand/modifier(s). In this embodiment, the method results in a decrease of a viability characteristic or quality and/or quantity of viable sperm produced by the subject and contacted with the ligand/modifier(s).

The targeted T1R3 and/or gustducin and/or cAMP and/or their respective pathway intermediates selected for use in this method may that those of the mammal being treated or homologs or orthologs thereof. Similarly the ligand/modifier(s) selected are those that bind the specific T1R3 and/or gustducin and/or cAMP and/or their respective pathway intermediates, homologs, and orthologs for the mammalian subject and that have the desired effect on the targets.

In another aspect, a contraceptive method can employ such a contacting step in a female mammalian subject by contacting sperm cells present in the reproductive system of a female prior to fertilization with a sufficient amount of the specified ligand/modifier(s) or compositions resulting in a decrease of a viability characteristic or quality and/or quantity of viable sperm. In such an embodiment, the ligand/modifier(s) is administered intravaginally prior to fertilization, such as by a douche, vaginal treatment, or contraceptive treatment, to reduce fertility. In another embodiment, the ligand/modifier(s) can be present in a contraceptive composition introduced into the female reproductive system prior to fertilization, or prior to intercourse.

In these contraceptive methods, the targeted T1R3 and/or gustducin and/or cAMP and/or their respective pathway intermediates selected for use in this method may that those of the mammal being treated or homologs or orthologs thereof. Similarly the ligand/modifier(s) selected are those that bind the specific targets, homologs, and orthologs for the mammalian subject and have the appropriate effect thereon. In one embodiment, the method results in a decrease of the quality and/or quantity of sperm.

The embodiments of the methods for decreasing fertility or contraception can be used for human contraception and for use in control animal populations, particularly pest populations. The differences in sensitivity to the selected ligand/modifier(s) among animal species enables one to design a ligand/modifier(s) composition that can be used to differentially control spermatogenesis or spermiogenesis in one species (i.e., as a contraceptive) without affecting others. This is particularly advantageous in the wild.

B. Methods for Enhancing Fertility

In an aspect of the invention in which the goal is to enhance fertility or treat hypofertility or suboptimal spermatogenesis or spermiogenesis, the method involves administration to a male subject of a composition or ligand/modifier(s) or molecule, as defined in detail above, that provides, increases or up-regulates the activation, expression, signaling or activity of a T1R3 receptor or an intermediate in its signaling pathway. In another embodiment, the method involves administering to a male subject a composition or ligand/modifier(s) or molecule that provides, increases or up-regulates the activation, expression, signaling or activity of G-α-gustducin, or a signal pathway intermediate thereof. In yet another embodiment, a fertility enhancing method involves administering to a male subject a composition or ligand/modifier(s) or molecule that decreases, down regulates, inhibits or blocks the activation, expression, signaling or activity of cAMP or a signal pathway intermediate thereof. In one aspect, the latter ligand/modifier(s) is a PDE activator. In another aspect, the latter ligand/modifier(s) is a PKA inhibitor. Such compositions, compounds or molecules may include any of those identified in detail above which have the intended effect on the targeted T1R3 and/or gustducin and/or cAMP and/or their respective pathway intermediates. In this embodiment, administration can be direct, systemic or local to the male subject as discussed above.

In this embodiment, the method enhances fertility of the subject by enhancing spermatogenesis or spermiogenesis, the quality or quantity or a viability characteristic of sperm produced by the male subject.

In still another embodiment, a method of enhancing spermatogenesis or spermiogenesis or fertility in a male mammalian subject involves contacting the subject's testis cells, germ cells or sperm with a sufficient amount of a composition or ligand/modifier(s) or molecule, as defined in detail above, that increases or up-regulates the activation, expression, signaling or activity of the T1R3 receptor or its signal pathway intermediates expressed on or in the cells. In still another embodiment, a method of modifying fertility in a male mammalian subject involves contacting the subject's testis cells, germ cells or sperm with a sufficient amount of a composition or ligand/modifier(s) or molecule, as defined in detail above, that increases or up-regulates the activation, expression, signaling or activity of G-α-gustducin, or its signal pathway intermediates expressed on or in the cells. In still another embodiment, a method of modifying fertility in a male mammalian subject involves contacting the subject's testis cells, germ cells or sperm with a sufficient amount of a composition or ligand/modifier(s) or molecule, as defined in detail above, that decreases, down regulates or inhibits the activation, expression, signaling or activity of cAMP or its signal pathway intermediates. According to this embodiment, the contacting of the selected ligand/modifier(s) and the target receptor, gustducin, cAMP or respective pathway intermediates expressed in or on the cells or sperm occurs ex vivo. This method includes treating a sample of a mammalian subject's sperm with a sufficient amount of the specified ligand/modifier(s). In this embodiment, the method results in an increase of a viability characteristic or quality and/or quantity of viable sperm produced by the subject and contacted with the ligand/modifier(s). Such contact between the ligand/modifier(s) and the targeted receptor, gustducin, cAMP or respective pathway intermediates in or on the sperm cells can occur during collection, treatment, storage, transportation, or administration of sperm as part of an in vitro fertilization (IVF) procedure. For example, a suitable dosage of the ligand/modifier(s) can be introduced into the medium in which the sperm is collected and used for insemination.

The targeted T1R3 and/or gustducin and/or cAMP and/or their respective pathway intermediates selected for use in this method may that those of the mammal being treated or homologs or orthologs thereof. Similarly the ligand/modifier(s) selected are those that bind the specific T1R3 and/or gustducin and/or cAMP and/or their respective pathway intermediates, homologs, and orthologs for the mammalian subject and that have the desired effect on the targets.

In still another aspect, a method of modifying fertility in a female mammalian subject employs the selected ligand/modifier(s) identified immediately above in the male fertility enhancement method. In the case of the female subject, fertility can be modulated by administering to a female subject a sufficient amount of the ligand/modifier(s) to the targets expressed on sperm that are present in the reproductive system prior to fertilization. Contacting sperm cells present in the reproductive system of a female prior to fertilization with a sufficient amount of the specified ligand/modifier(s) or compositions defined herein can result in an increase of a viability characteristic or quality viable sperm. In one embodiment, the contacting occurs during collection, treatment, storage, transportation, or administration of sperm as part of an in vitro fertilization (IVF) procedure. Alternatively, the contact between the ligand/modifier(s) and the sperm can occur any time before fertilization, and thus the ligand/modifier(s) can be introduced into the female reproductive system is present in a douche or vaginal treatment administered prior to or during insemination or IVF.

In the various embodiment, such contact increases spermatogenesis or spermiogenesis in the male, and can increase the quality and/or a viability characteristic of sperm and such method is desired wherein fertility in the subject is compromised or where an increase in fertility is desired. The embodiments for increasing fertility can be used for human IVF and also for animal breeding purposes. The differences in sensitivity to the selected ligand/modifier(s) among animal species enables one to design a ligand/modifier(s) composition that can be used to differentially increase spermatogenesis or spermiogenesis in one species without affecting others. This is particularly advantageous in the animal breeding or in farming.

C. Screening Methods for Various Ligand/Modifier(s) Useful in the Treatment Methods or Test Molecules that Affect Spermatogenesis or Spermiogenesis

A method for screening a test molecule for its effect on fertility or spermatogenesis or spermiogenesis comprises contacting a mammalian sperm cell, testis cell or cell line expressing at least one of the targeted T1R3 and/or gustducin and/or cAMP and/or their respective pathway intermediates in vitro with a test molecule. In another embodiment, a method for screening a test molecule for its effect on fertility or spermatogenesis or spermiogenesis comprises contacting a mammalian sperm cell, testis cell or cell line that does not express at least one of the targeted T1R3 and/or gustducin and/or cAMP and/or their respective pathway intermediates in vitro with a test molecule. In one aspect, the cell or cell line contains the chimeric T1R3 receptor discussed in the examples. In another aspect, the cell or cell line expresses or does not express G-α-gustducin. In another embodiment, the cell is sperm from a single subject, or another germ cell, or from a pool of multiple subjects.

In still another aspect, the methods for screening enable the detection of a compound or molecule that can cross the blood-testis barrier to have its biological effect. The detection of this characteristic of a molecule or compound is another novel aspect of the screening methods described herein.

Thereafter, the method involves an assaying step comprising assaying the contacted cell or cell line for a change indicative of inhibition, activation, change in expression, signaling or activity of at least one of the targets in comparison with a reference cell or cell line contacted with a control molecule. A change a physical or functional characteristic of the test cell or cell line in comparison to a reference indicates a modifying effect of the test molecule on the quality, quantity or a viability characteristic of the sperm or spermatogenesis or spermiogenesis.

One of skill in the art can also recombinantly generate a cell or cell line that expresses a selected target from a male germ cell or from a cell that normally does not express that target by use of now-conventional techniques, or vice versa, as employed in creating the transgenic animals used in the examples below. However, the ready availability of sperm samples expressing the targeted T1R3 and/or gustducin and/or cAMP and/or their respective pathway intermediates or not expressing one or more of these targets would facilitate an assay for suitable ligand/modifier(s) in vitro in the absence of human subjects or animals, domestic or wild. The sperm cells can also be used to test the side effect of known medications used as test molecules in an assay on fertility or spermatogenesis or spermiogenesis, e.g., viability of sperm.

In certain embodiments, the screening method is a high-throughput method comprises multiple cells, cell lines, test molecules and references. In still other embodiments, the assaying step comprises an imaging assay or any of the assays for characteristics as disclosed in detail in the examples below.

The test molecule is selected from among known or unknown ligand/modifier(s) of the target T1R3 and/or gustducin and/or cAMP and/or their respective pathway intermediates, homologs or orthologs. Alternatively the test molecule is selected from among libraries of molecules of unknown function or known medications to assess the effect of the test molecule on the physical or functional characteristics of normal sperm. The cells once contacted for a suitable time with a test molecule are then assayed for a change in a physical or functional characteristic of the cells in comparison with a reference cell or cell line contacted with a control molecule or contacted with nothing at all. A change in a physical or functional characteristic of the test cells or cell lines vs. the reference indicates a modifying effect of the test molecule on the quality or viability of the sperm.

For example, the contacted cell or cell line may be assayed for a change in the cytosolic calcium concentration indicative of T1R3 receptor activation in comparison with a reference cell or cell line contacted with a control molecule or with nothing. An increase or decrease in cytosolic calcium concentration of the test cell or cell line vs. the reference indicates a modifying effect of the test molecule on the quality and/or quantity of sperm or some other characteristic of spermatogenesis or spermiogenesis. Other characteristics such as motility, viability, etc of sperm can also be evaluated in such an assay. In another embodiment, such assays can include electrophysiological responses or calcium imaging of isolated testes cells, cell lines or sperm cells.

These methods may also be high-throughput screening methods. In one embodiment such an assay involves contacting in each individual well of a multi-well plate a different selected test molecule (e.g., ligand/modifier(s), known or unknown molecules or drugs) with a mammalian germ cell or cell line (e.g., sperm) that expresses or lacks expression of one or more the above-referenced targets. After the test molecule has been exposed to the germ cell, testes cell or sperm cell under appropriate culture conditions, a physical or functional characteristic of the cell is conventionally measured. A change in a characteristic of the cell caused by any of the test molecules vs. the control permits the selection of the test molecule as one which enhances fertility or decreases fertility.

Such other assay formats may be used and the assay formats described herein are not a limitation. In one embodiment an acrosome reaction assay may be used. Whether a compound can increase or decrease acrosome reaction is important to the success of fertilization, particularly for IVF. Such assays are useful to identify useful ligand/modifier(s), as well as to distinguish between those that enhance the quality, viability and/or quality of sperm from those that have the opposite effect. Other assays useful in these methods are immunocytochemistry assays, immunostaining assays, Western blots, TUNEL assays, cholesterol depletion assays, and cAMP concentration or activity assays, among others.

Another form of screening is to design a compound which has structural similarity to the various target ligand/modifier(s) identified herein by computational evaluation and a series of steps in which chemical entities or fragments are screened and selected for their ability to associate with or mimic known compounds or test molecules or ligand/modifier(s) described herein. One skilled in the art may use one of several methods to screen chemical entities or fragments for their ability to mimic the structure of these peptides and more particularly to identify the structure that binds with the targeted T1R3 and/or gustducin and/or cAMP and/or their respective pathway intermediates. This process may begin by visual inspection of, for example, a three dimensional structure on a computer screen. Selected fragments or chemical entities may then be positioned in a variety of orientations to determining structural similarities, or docked, within a putative binding site of the receptor.

Specialized computer programs that may also assist in the process of designing new test molecules based on those identified by the methods herein include the GRID program available from Oxford University, Oxford, UK.; the MCSS program available from Molecular Simulations, Burlington, Mass.; the AUTODOCK program available from Scripps Research Institute, La Jolla, Calif.; and the DOCK program available from University of California, San Francisco, Calif., and software such as Quanta and Sybyl, followed by energy minimization and molecular dynamics with standard molecular mechanics force fields, such as CHARMM and AMBER. Additional commercially available computer databases for small molecular compounds include Cambridge Structural Database, Fine Chemical Database, and CONCORD database, among others.

Once suitable chemical entities or fragments have been selected, they can be assembled into a single compound. Assembly may proceed by visual inspection of the relationship of the fragments to each other on the three-dimensional image displayed on a computer screen in relation to the structure of the receptor. Useful programs to aid one of skill in the art in connecting the individual chemical entities or fragments include the CAVEAT program, 3D Database systems such as MACCS-3D database (MDL Information Systems, San Leandro, Calif.); and the HOOK program, available from Molecular Simulations, Burlington, Mass.

Compounds that mimic a test molecule described herein may be designed as a whole or “de novo” using methods such as the LUDI program available from Biosym Technologies, San Diego, Calif.; the LEGEND program available from Molecular Simulations, Burlington, Mass.; and the LeapFrog program, available from Tripos Associates, St. Louis, Mo. Other molecular modeling techniques may also be employed. For example, where the structures of test compounds are known, a model of the test compound may be superimposed over the model of the target receptor. Numerous methods and techniques are known in the art for performing this step, any of which may be used. The model building techniques and computer evaluation systems described herein are not a limitation.

Thus, using these computer evaluation systems, a large number of compounds may be quickly and easily examined and expensive and lengthy biochemical testing avoided. Moreover, the need for actual synthesis of many compounds is effectively eliminated.

### III. COMPOSITIONS

In one embodiment, a contraceptive composition for decreasing spermatogenesis or spermiogenesis or fertility in a mammalian subject comprises a ligand/modifier(s) or reagent that decreases, inhibits, or down-regulates the activation, expression, or activity of one or more T1R3 receptors or an intermediate in the signaling pathways thereof in the subject's testis cells, germ cells or sperm. In another embodiment, a contraceptive composition for decreasing spermatogenesis or spermiogenesis or fertility in a mammalian subject comprises a ligand/modifier(s) or reagent that decreases, inhibits, or down-regulates the activation, expression, or activity of G-α-gustducin, or an intermediate in the signaling pathways thereof in the subject's testis cells, germ cells or sperm. In still another embodiment, a composition comprises a pharmacological agent or ligand/modifier(s) that blocks T1R3 and gustducin function or their signaling pathways respectively. In some embodiments, the ligand/modifier(s) acts on an intermediate of T1R3 or gustducin, such as PDE. In still another embodiment, the composition contains a ligand/modifier(s) or reagent that increases, activates or up regulates the activation, expression, signaling or activity of cAMP.

In one example, this composition comprises a ligand/modifier that is a T1R3 receptor inhibitor, as exemplified above, and a ligand/modifier that is a gustducin inhibitor. In another embodiment, the composition comprises a combination of a T1R3 inhibitor and an inhibitor of PDE which is normally activated by gustducin.

Because T1R3 and gustducin exert their negative reproductive activities through elevation of cAMP (potentially also cGMP), any agent that is known to increase cAMP can be used in addition in the compositions described herein. An agent that increases cAMP (e.g., adding forskolin available as a food supplement to the dual T1R3/PDE blocker) would greatly enhance the contraceptive effect on spermatogenesis or spermiogenesis. In one embodiment, the composition contains an additional agent that increases cAMP added to the combination of a T1R3 receptor inhibitor and/or gustducin inhibitor. The addition of the cAMP ligand/modifier(s) may allow for reduction of the concentration of the inhibitors and reduce their side-effects.

In still another embodiment the composition contains a cAMP activating agent, a T1R3 inhibiting ligand/modifier(s), and optionally a gustducin inhibiting ligand/modifier(s) and optionally a PDE inhibiting ligand/modifier(s). In still another embodiment, the composition contains a PKA activator. It is particularly desirable if one or more of the ligand/modifier(s) is specific to testis cells.

In another embodiment, a composition for increasing fertility in a mammalian subject comprises a ligand/modifier(s) or reagent that increases, activates, or up-regulates the activation, expression, or activity of one or more T1R3 receptors or an intermediate in the signaling pathways thereof in the subject's testis cells, germ cells or sperm. In another embodiment, a composition for increasing fertility in a mammalian subject comprises a ligand/modifier or reagent that increases, activates, or up-regulates the activation, expression, or activity of G-α-gustducin, or an intermediate in the signaling pathways thereof in the subject's testis cells, germ cells or sperm. In still another embodiment, a composition optionally includes a reagent that decreases, inhibits, or down-regulates the activation, expression, signaling or activity of cAMP or a signal pathway intermediate thereof.

In one embodiment, a composition comprises a pharmacological agent that activates T1R3 and gustducin pathways to improve male fertility. In one embodiment, this agent comprises direct activators of T1R3 and gustducin or of activators of T1R3 and gustducin signaling pathways. Any of such molecules could potentially co-stimulate T1R3 and or gustducin pathways and improve male fertility, particularly in cases where male fertility is due to block in these molecules' signaling.

Because T1R3 and gustducin exert their activities in reproduction mainly through modulation of cAMP, any agent that is known to decrease cAMP can be used in addition. Such an agent may be also e.g. a PDE activator, and/or a PKA blocker. Finally, the ligand/modifier(s) described herein can be added in suitable dosages to the culture media used to collect, store, transport or administer sperm for insemination or IVF procedures.

As noted above with respect to either of the contraceptive or fertility enhancing compositions methods, the selection of ligand/modifier(s) employed in the compositions may be made from those known ligand/modifier(s) identified above, from other ligand/modifier(s) that are newly identified to have such activity and/or from newly identified ligand/modifier(s) identified in the screening methods. The compositions as described herein may employ ligand/modifier(s) that are specifically active in a selected species of mammalian subject. Human and other mammalian T1R3 receptors, gustducin, cAMP and/or their pathway intermediates may be sufficiently different that human-specific ligand/modifier(s) that inhibit or activate them have been identified. Other species-specific ligand/modifier(s) can be developed for species-specific contraception or breeding enhancement. Such compositions can be used to effect male specific and reversible sterility or alternatively enhance fertility as desired.

Various ligand/modifier(s) and combination of ligand/modifier(s) can be further screened to determine if the inhibition of spermatogenesis or spermiogenesis produces a short term effect, permitting use as a male non-hormonal contraceptive, such as one day (evening) pill. Such ligand/modifier(s) and/or combinations thereof can be used as a female non-hormonal contraception pill if the ligand/modifier(s) are small molecules capable of distributing in biological fluids and thus could interfere with the sperm motility, capacitation or fertilization.

The compositions identified above may be a topical cream or ointment containing not only a suitable dosage of the ligand/modifier(s), but also conventional pharmaceutical excipients and other conventional ingredients. Another embodiment of a composition for administration to a male subject is a transdermal patch in which the ligand/modifier(s) are embedded in a suitable dosage for topical delivery over a period of time. Still another embodiment is an implant that is designed to be located close to the testicles and which permits timed release in vivo of the ligand/modifier(s) into the blood stream of the male subject.

Compositions for in vivo use in the female reproductive tract can be a rinse, douche or vaginal créme or ointment containing ligand/modifier(s) that affects the quality of sperm prior to fertilization or intercourse. Conventional pharmaceutical excipients and other conventional ingredients common to compositions for intravaginal use and not incompatible with the ligand/modifier(s) are also provided by these compositions.

In the contraceptive compositions the ligand/modifier(s) used are those that decrease spermatogenesis or spermiogenesis or the quality and/or quantity of sperm produced by a male subject or present in a female reproductive tract prior to fertilization. One such embodiment is a contraceptive device which is impregnated with, or coated with, a suitable amount or dosage as described above, of one or more suitable ligand/modifier(s). For example, the ligand/modifier(s) can be present in effective amounts and coated onto a condom designed for males or females. In another the ligand/modifier(s) can be present in effective amounts in conventional contraceptive compositions, with or without additional contraceptives, such as spermicides. Similarly, the ligand/modifier(s) can be coated onto a contraceptive implant or device, such as an IUD or contraceptive sponge. Alternatively, the composition for intravaginal contraceptive administration to a female subject may contain the ligand/modifier(s) present in an amount sufficient to decrease the quality and/or quantity of sperm with which it comes in contact. Such a composition may also contain other known contraceptive agents.

For fertility enhancement, the compositions may be for administration to a male subject to enhance spermatogenesis or spermiogenesis. Alternatively, the ligand/modifier(s) identified herein may be useful in compositions for collecting, storing or transporting or administering sperm for insemination or IVF procedures, or may be present in a rinse, douche or vaginal treatment for use in a female prior to fertilization or intercourse.

The compositions containing ligand/modifier(s) described herein for contraception or enhancing spermatogenesis or spermiogenesis or fertility may be formulated neat or with one or more excipient for administration. One of skill in the art would readily be able to determine suitable excipients based on the identity and number of selected ligand/modifier(s), the number of components of the composition, the mammalian subject, the purpose for the composition, dosage needed and administration route, among others. Not only may the composition be solid or liquid, but excipient(s) may be solid and/or liquid carriers. The carriers may be in dry or liquid form and must be pharmaceutically acceptable. The compositions are typically sterile solutions or suspensions suitable for the mode of administration.

When the route of administration is intravenous injection or implant, the composition may be in the form of a liquid or suspension. When the route of administration is topical, such as transdermal patch, the composition may be additionally in the form of a créme. All known routes of administration may be used, although local administration may be desirable. One of skill in the art would readily be able to formulate the compositions discussed herein in any one of these forms.

Suitable carriers and excipients include liquid carriers that may be utilized in preparing solutions, suspensions, and emulsions. In one embodiment, at least one ligand/modifier(s) is dissolved a liquid carrier. In another embodiment, at least one ligand/modifier(s) is suspended in a liquid carrier. In one embodiment, the liquid carrier includes, without limitation, water, organic solvents, oils (such as fractionated coconut oil, arachis oil, corn oil, peanut oil, and sesame oil and oily esters such as ethyl oleate and isopropyl myristate), fats, cellulose derivatives such as sodium carboxymethyl cellulose.

Examples of excipients which may be combined with the ligand/modifier(s) include, without limitation, antioxidants, binders, buffers, coatings, coloring agents, compression aids, diluents, disintegrants, emulsifiers, emollients, encapsulating materials, fillers, glidants, granulating agents, lubricants, metal chelators, osmo-regulators, pH adjustors, preservatives, solubilizers, sorbents, stabilizers, surfactants, suspending agents, thickening agents, or viscosity regulators. Other excipients may be used as listed in a variety of references. Specific examples of excipients for use in solid formulations include, without limitation, calcium phosphate, dicalcium phosphate, magnesium stearate, talc, starch, sugars (including, e.g., lactose and sucrose), cellulose (including, e.g., microcrystalline cellulose, methyl cellulose, sodium carboxymethyl cellulose), polyvinylpyrrolidine, low melting waxes, ion exchange resins, and kaolin. In one embodiment, the pharmaceutically acceptable excipient is a surfactant, binder, coating, disintegrant, filler, diluent, flavouring agent, coloring agent, lubricant, glidant, preservative, sorbent, sweetener, solubility increaser (such as cyclodextrans), analgesia enhancer (such as caffeine), or a combination thereof

### IV. Examples

Most of the spermiogenic genes are highly conserved between mice and human. Therefore rodent studies not only reveal essential roles of various genes in multiple developmental stages of haploid male germ cells, but also point to the notion that these genes and their products are good targets for male contraceptives (Ref 6). Mouse models are selected for these experiments because they are the only practical species in which transgenic animals can be generated. Mice have a relatively short gestation period and high fecundity and are very well characterized genetically. The mouse provides a unique animal model in which to study developmental and biochemical aspects of male reproduction as well as the effects of a variety of chemical agents on reproduction.

Examples 1-13 provide evidence that a binary agent that blocks both human T1R3 and α-gustducin or their downstream pathways elicits profound effects on male reproduction. These results lead to methods and the identification of new compositions that disrupt spermatogenesis or spermiogenesis and, conversely, to the development of novel male contraceptives.

### Example 1

### Mouse Models Expressing Humanized T1R3

To get insights into the function of the human receptor in vivo we generated transgenic animals expressing a humanized form of T1R3.

Mouse models that lack only T1R3 (described as T1R3 knock-out, T1R3 KO, or T1R3-null) or only gustducin (gustducin knock-out, or gustducin-null) are known. They reproduce normally.

A. Line 7 Mouse Model—

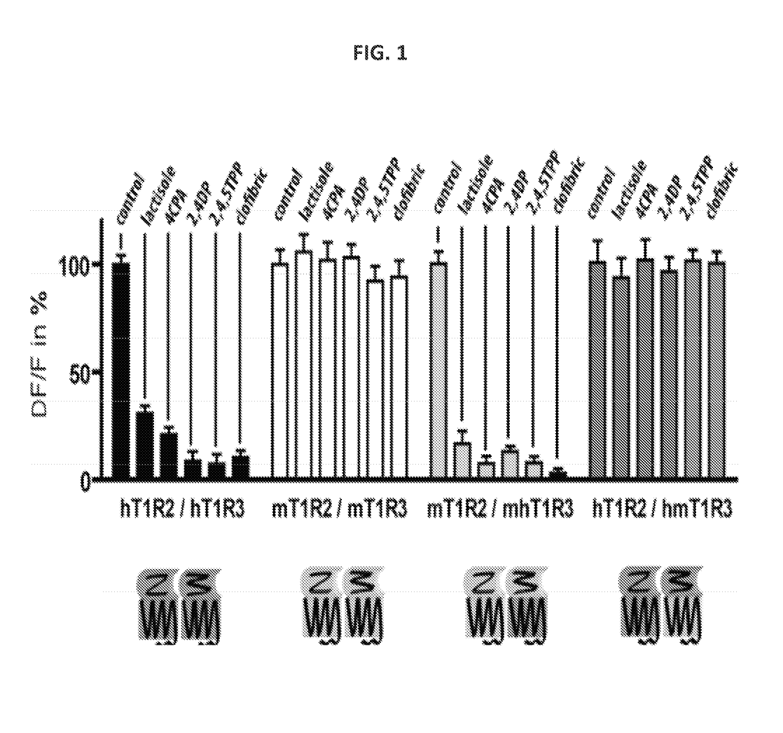

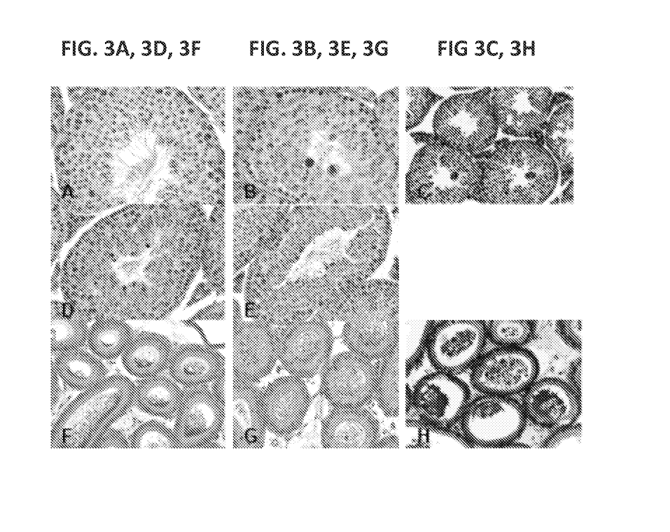

A mouse model was developed that expresses a human version of the T1R3 receptor. The humanized receptor and its function in vitro was published (Maillet et al, cited above). The humanized receptor is a mouse-human chimera, where the extracellular domain is of mouse origin and the transmembrane and intracellular domains of human origin. The transmembrane portion of human T1R3 is targeted by a number of chemicals, including lactisole, the widely used phenoxy auxin herbicides, and anti-lipidemia fibrate drugs such as clofibric acid, which potently inhibit human but not rodent sweet (T1R2+T1R3) receptors (Maillet, cited above; FIG. 3). In vitro structure-function studies have shown that the full-length human T1R3 does not form a sweet receptor when combined with mouse T1R2. In contrast, the chimeric mhT1R3 receptor, having a mouse extracellular domain and a human transmembrane domain, pairs with mouse T1R2 receptors to form a fully functional sweet receptor that is inhibited by lactisole and other phenoxy compounds. The chimeric mhT1R3 receptor shown in FIG. 1 (underlined) was used to produce transgenic animals.

We used the chimeric mhT1R3 receptor to produce conventional transgenic animals. The DNA construct was injected into a genetically engineered mouse line that lacks endogenous T1R3 (T1R3KO or T1R3-null). The DNA construct contained the coding region of the chimeric mhT1R3 receptor preceded by 13 kb of T1R3 promoter (used previously in another study (Damak S, et al., 2008 BMC Neurosci 2; 9:96) and followed by the IRES (internal ribosomal entry site) sequence and eGFP (enhanced green fluorescent protein) gene (Wilson D A, et al 2009 Ann N Y Acad Sci. 1170, Suppl 1:1-11). Therefore, only the humanized receptor is present in the mouse and any responses known to be due to T1R3 stimulant must reflect only the function of this human type receptor. The mice expressed the humanized T1R3 faithfully in all tissues where T1R3 is normally expressed, including testis. Thus these mice can be used as model organisms to mimic human taste and biochemical responses to various agonists and inhibitors of T1R3. We call these mice Line7.

We established two lines, line 7 and line 9, with two and six copies of the mhT1R3 transgene, respectively. For further crosses, we used the line 7 that has fewer copies of the transgene. In contrast to the nontransgenic KOs, transgenic animals from both lines have preferences for sugars and sweeteners, which directly indicate that the mhT1R3 transgene is expressed and functional in the taste system. The transgenic mice responded to sugar and artificial sweeteners as did WT mice, but the responses were effectively blocked by phenoxy compounds.

In contrast to mouse T1R3 or WT mice, the humanized receptor can be pharmacologically manipulated, e.g., blocked by human-specific inhibitors such as lactisole, fibrates and phenoxy herbicides. Line 7's sweet preference was blocked by 1 mM clofibric acid and 0.2 mM 2,4-DP as was shown in vitro with the chimeric mhT1R3 receptor. Therefore, we have verified that the chimeric receptor is expressed and functions like the human receptor both in vitro and in vivo. This is in contrast to WT mice and probably all rodents and many other species, as their receptors are insensitive to this inhibition. Based on available data, humans, old world monkeys and primates have T1R3 receptors that are blocked by phenoxy compounds.

B. Line 7c Mouse Model—

Line7 animals were crossed with gustducin-null animals, producing this complex genotype (T1R3−/−, Gus−/−, mhT1R3+/−), i.e., animals that lack gustducin, lack endogenous T1R3, and express only the humanized T1R3 susceptible to inhibition. That these animals were generated at all further validates the proper expression of the mhT1R3 transgene. In the absence of the transgene, we were unable to obtain double-nulls. We called the T1R3−/−, Gus−/−, mhT1R3+/− animals “Line 7c” for simplicity. They were healthy and fertile. In histological examinations we found no pathological signs in sections of testis and epididymis (data not shown).

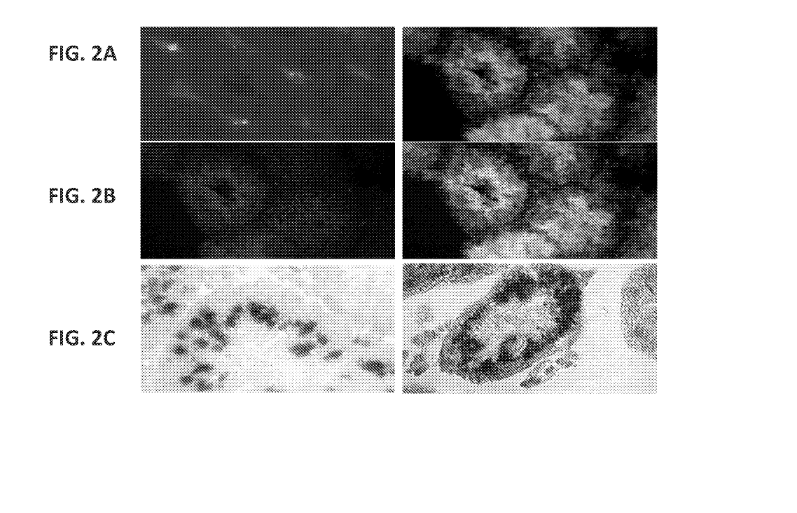

In transgenic lines 7, 9 and 7c, we have verified the expression of the mhT1R3 transgene in taste cells and testes (Wilson, cited above; FIG. 4). Furthermore, our preliminary data showed endogenous T1R3 mRNA and endogenous α-gustducin protein expression in haploid spermatids in testicular tubules (FIG. 2), in agreement with published reports that T1R3 is expressed in testis and α-gustducin is expressed in testis and sperm. Importantly, the same cells express T1R3, α-gustducin, and CREM (Blendy et al, cited above).

The transgenic animals from Line 7, 9 or 7c can be used to screen compounds that interact with T1R3 alone or with T1R3 in a complex with other receptors and have an effect on reproduction. Such compounds may be produced normally in organs (e.g. in testis) or chemically synthesized. The humanized receptor in Lines 7, 9 and 7c was expressed in taste cells together with associated GFP reporter. Transgenic animals showed strong preference to sucrose, and modest preference to the artificial sweetener sucralose. In contrast to WT animals they also displayed preference for the human-specific sweetener cyclamate. The sweet responses of the transgenic animals were completely blocked by clofibric acid, a human-specific inhibitor that has no effect on the mouse receptor. Other experiments showed that widely used phenoxy herbicide 2,4DP, also specific to human receptor, was an even more powerful inhibitor in the humanized mouse than clofibric acid.

In contrast to the endogenous T1R3 in WT animals, the humanized T1R3 in transgenic animals appeared to be able to partially rescue the male-specific transmission ratio distortion. T1R3/α-gustducin double-null animals with the humanized T1R3 transgene (mhT1R3) were fertile.

We placed Line 7c (gustducin null animals expressing the humanized T1R3) on a diet containing a T1R3 inhibitor (prodrug), clofibrate. We found similar pathology as in CREM KO and in gustducin/T1R3 double KO animals in testis. In contrast to CREM and DKOs these animals still produced about ¼ of sperm normally produced by WT males. However, functionally these treated males were sterile, unable to fertilize females and to produce offspring. We concluded that in clofibrate treated animals the T1R3 inhibition was only partial. The onset of sterility in males on clofibrate diet has been shorter than originally thought, perhaps as short as 1 week or 10 days and it was fully reversible. The block with clofibrate should be specific to humans and primates.

The major defect appears to be at the level of developing spermatids. Our preliminary data indicated that blocking T1R3 and α-gustducin did not result in an obvious pathology in any organs other than testis.

C. DKO Line—

While breeding the transgenic Line 7c, we found on very rare occasions T1R3/α-gustducin double-nulls without the mhT1R3 transgene (DKO). So far we have obtained two animals with this genotype, and fortunately both were males. We speculate that the transgenically expressed humanized mhT1R3 in the form of either RNA or protein can more readily diffuse among the joined spermatids and therefore rescue the TRD. The two males were of normal size, indistinguishable from their peers, without any obvious pathology. Their testes were of a normal size. However, the vas deferens and cauda epididymis contained no living sperm. The DKO males produced no viable sperm and are sterile. Upon biopsy there was cellular debris in the tubules that fell apart after extraction. The volume of this material was about the same as that of the sperm-containing material from WT males.

In summary, we have found that gustducin, T1R3 and CREM are all expressed in the same testicular cells. CREM−/− females are fertile. We found in intercrossing of T1R3 and gustducin null animals that male germ cells lacking both T1R3 and gustducin (Line 7c) were not transmitted to progeny, and uncovered male specific transmission ratio distortion (TRD).

### Example 2

### Methods Used in the Examples

A. T1R3 Inhibitors.—

The potent human T1R3 inhibitors clofibric acid and 2,4-DP have IC₅₀ values of 28 and 6 nM, respectively. We used clofibrate in our experiments because it is an approved medication for human use and numerous human and animal preclinical and research studies have used it (e.g., Staels, B., and Fruchart, J. C. 2005 Diabetes 54, 2460-2470.). Clofibrate is an esterified prodrug, converted into the actual inhibitor, clofibric acid, after its absorption in vivo. Unlike clofibric acid, clofibrate has no effect on taste, so it is an ideal compound to use in a diet.

B. T1R3 Pharmacological Block.—

Matched groups (six males each) of WT and line 7c mice (expressing the chimerical mhT1R3 transgene on a T1R3/α-gustducin double-null background), 3-6 months old, were fed the indicated diets for 4-6 weeks. Each genotype was tested in two groups, one on the regular diet and one on a test diet with clofibrate. An additional group of males stayed on the test diet for another 4-6 weeks, during which time they were mated with WT females to determine whether the pharmacological intervention was effective. Then the animals were allowed to recover on the regular diet for an additional 4 weeks after the end of the treatment (half-time for total sperm turnover in mice), and again were tested for their reproductive capacity, and then their tissues were analyzed. The diets were normal chow and normal chow with clofibrate (250 and 500 mg/kg body weight daily). The dosage of clofibrate was comparable to that used in human treatment (Staels, cited above) and animal studies. As additional controls we treated T1R3-null mice, α-gustducin-null mice, and mice expressing the humanized mhT1R3 receptor on a WT α-gustducin background with clofibrate. T1R3 and α-gustducin single-null animals were fertile, and we do not anticipate an effect of the human-specific inhibitor on rodent receptors. Therefore, we tested these animals only for the 4 weeks without the recovery period.

C. PDE Pharmacological Block.—

Matched groups of WT and T1R3-null animals, 3-6 months old, had theophylline (1.5 mg/ml) in their drinking water for 4 weeks. We followed protocols from published rodent studies (e.g., Morrissey R E, et al., 1988 Fundam Appl Toxicol. 10(3):525-36).

D. T1R3 and PDE Block.—

We used our transgenic mice expressing the humanized mhT1R3 receptor in the T1R3-null/α-gustducin WT background generated as described above. Matched groups of WT and mhT1R3 mice 3-6 months old had clofibrate in their diet and theophylline in drinking water for 4 weeks. Control groups of mice with T1R3-null, α-gustducin-null, or humanized mhT1R3 in WT and T1R3-null backgrounds without any treatments were used as additional controls. We have preliminary data from a few animals with these genotypes showing that there is no detectable pathology in testis after a single block with either clofibrate or theophylline. Animals from all these lines were also fertile.

E. Clofibrate Diets.—

The rapid metabolism of clofibric acid required human patients to take the medication every 6 hours. Because such a feeding interval cannot be readily achieved with mice through dietary consumption, we suspect that even with high doses in food, the blood level fluctuates and therefore the inhibition in our preliminary experiments was only partial. We think it was enough to produce functional sterility, but not enough to block all spermatogenesis or spermiogenesis. Delivery by osmotic minipump may overcome this limitation; however we need 3 mg clofibrate, which has poor solubility (5 mg/ml), for a daily dose. A better approach to achieve more complete pharmacological block is to use a drug sustained-delivery mechanism. Recently a number of methods were developed to alter properties of drugs so that they were slowly dissolving in the gut and produce relatively steady active blood levels after a one-day or even one-week dosing (83,84). Most frequently, poly(lactic-co-glycolic acid) (PLGA) particles were used. Some of the protocols to produce drug-containing PLGA particles were simple to follow, and therefore we produce clofibrate in this sustained-release form. Another alternative is to use an automated syringe pump used for human i.v. infusions that can deliver an accurate dose of a drug at a set volume and interval. Such an instrument can be attached to a cage and deliver the drug in the form of a palatable paste at set intervals.

F. Theophylline Treatment.—

There was no obvious adverse effect on health and behavior of treated animals. Our preliminary data showed that testes in T1R3-null but not in WT animals display similar, although milder, pathology as those in clofibrate-treated animals (data not shown).

G. Cell Isolation, Cell Sorting.—

For in vitro experiments we isolated tubular cell types using published methods (e.g., La Salle S, et al., 2009 Methods Mol Biol 558:279-97). In addition, we sorted the GFP-positive cells (GFP is expressed from the mhT1R3 transgene) using a FACS cell sorter. We have isolated testicular cells with collagenase and liberase and found the protocol comparable to our routine procedures for pancreatic islet isolations. We used the same tissues and sorted cells here and also for additional experiments proposed.

H. Anatomy.—

We analyzed the testicular and epididymal size and weight, and noted any morphological differences between mutant and WT animals.

I. Histology and Immunohistochemistry.—

For histological analyses of testicular structure in these mutant animals we used Bouin's or Davidson's fixatives and paraffin sections with H&E and PAS staining for fine tissue structure analysis and where possible for immunohistochemistry (see FIGS. 2 and 3). We used appropriate antibodies to determine the cell types (e.g., Tamoo et a; 2-2. Jostpcje, Ce;; bop;. 118(5):409-14). Measurement/staining of apoptotic cells was done with commercially available kits, such as TdT TUNEL enzyme (Roche). To analyze apoptosis in more detail, we used a kit from Cell Signaling Technology, Inc. (Danver, Mass.) that includes specific antibodies to inactive and active caspases. Histological, immunological and cellular analyses, and genetic determination of gene transmission, were also conducted using conventional techniques.

J. Hormone Levels.—

Testosterone, follicle-stimulating hormone, leutinizing hormone, and inhibin were measured using standard ELISA kits (Holzel Diagnostika, Cologne, Germany) and/or at the Core Biochemistry lab at UPenn.

K. Tissue Explants.—

Short term cultures or isolation of testicular cells assessed changes during T1R3 inhibitor treatment in vitro. We use the BSA gradient sedimentation method as described (La Salle, cited above).

L. Expression of CREM and Downstream Genes.—

We used RT-PCR and qRT-PCR to determine expression of CREM, Tnps, protamines, alspermine, Krox-20 and 24, and TISPs (transcription increased in spermatogenesis or spermiogenesis) and compared them with expression of genes not regulated by CREM such as acr, Hoxa-1, and G6PD. We identified CREM transcriptional variants and determined whether these encoded a repressor or an activator. We used qPCR to determine relative expression levels in the single-null animals and heterozygotes.

M. Sperm Evaluation.—

We observed spermatozoa after clofibrate treatment represent cells that were able to escape the inhibition of clofibric acid, most likely due to very fast clearance and fluctuating levels in rodents. This allows some tubular cells to complete the developmental cycle. Therefore, we measured sperm concentration as an indicator of efficiency of the pharmacological block and to be able to determine the concentration that results in effective sterility. We analyzed testicular morphology, sperm production, sperm motility and sperm function in in vitro fertilization after feeding these animals with the lipid lowering drug clofibrate. We tested an acute inhibition of sperm motility and function in vitro by incubating sperm from the engineered mice (mGus−/−, mT1R3−/−, hmT1R3+) in solutions containing clofibric acid or other T1R3 inhibitors. The concentration used was similar to the one used in human treatment with fibrates (˜0.03 g/kg weight, for 3 weeks). We tested sperm motility and function in vitro of the Gα-gustducin KO mice (missing only the Gα-gustducin gene (mGus−/−)) after blocking T1R3 receptors with the rodent specific inhibitor gurmarin (3 mg/ml).

### Example 3

### The Human Sweet Receptor is Inhibited by Lactisole and Other Phenoxy Compounds

The human T1R3 transmembrane domain determines sensitivity to lactisole, phenoxy herbicides, and fibrates. Inhibition of the receptor's response to sucralose occurred only in the presence of the transmembrane domain of human T1R3. We activated human (hT1R2/hT1R3), mouse (mT1R2/mT1R3), and chimeric mouse/human sweet receptors (as indicated by mh prefix) with 2.5 mM sucralose and assayed for inhibition by lactisole, phenoxy herbicides (chlorophenoxypropionic, dichlorophenoxypropionic, and trichlorophenoxy propionic acids), and a fibrate (clofibric acid), at ˜5× IC₅₀ in buffered saline. Chimeric receptors of T1R3 contained the extracellular portion of human or mouse receptor and the transmembrane domain and C-terminal domain from mouse or human receptor: mh indicates the presence of the mouse extracellular domain and human transmembrane domain; hm indicates the presence of the human extracellular domain and mouse transmembrane domain.

Specific inhibition by lactisole of sweet taste and the sweet receptor subunit T1R3 is well documented in the literature. We have also shown that a number of phenoxy compounds related to lactisole bind to the same region of the receptor. One of them, clofibric acid, has been used in humans for decades as an antilipid medication acting on PPAR-α with an EC₅₀ activity of 55 μM, and we found that it blocks the T1R3 receptor with an IC₅₀ of 28 μM. The phenoxy herbicide 2-(2,4-dichlorophenoxyl) propionic acid (2,4-DP) is even more active, with an IC₅₀˜5 μM (50). A more potent and/or specific inhibitor would be extremely helpful.

This example demonstrates that the fully human sweet receptor (T1R2+T1R3) is inhibited by phenoxy compounds. Mouse-human chimeric T1R3 is also inhibited, but not human-mouse T1R3. Our data indicated that the currently available T1R3 inhibitors were sufficient in combination with the humanized mhT1R3 transgene we have made and partially analyzed (see preliminary data FIG. 3).

We placed line 7c and WT males on a diet with clofibrate (300 mg/kg body weight), or a normal diet (no clofibrate), for 1 or 2 months. In the second month the males were housed with two young WT females to determine whether the males were fertile. The line 7c males on a regular diet were fertile. Although the clofibrate diet had no apparent effect on the health of any of the animals, during the second month on clofibrate the males from line 7c did not produce progeny, in contrast to WT males on clofibrate, which generated two litters each at that time. After 1 or 2 months of clofibrate treatment, the line 7c males had substantially less sperm (i.e., about one-quarter that of the WT males), whereas line 7c males on regular feed had the same amount of sperm as WT males. This example demonstrates that males with genetically ablated gustducin and mouse-humanT1R3 (line 7c) blocked pharmacologically with clofibrate produce about ¼ of normal number of sperm and were functionally sterile.

### Example 4

### Histology

Histology of Line 7c and WT males fed clofibrate showed pathological changes in testicular tubules and epididymi in line 7c males but not in WT males. The major pathological findings were the presence of giant cells in many testicular tubules and a lower number of spermatozoa in epididymis, with an abundance of immature cells and the presence of PAS positive material (FIG. 3). Histology of testes and epididymi from line 7c males fed regular diet (no clofibrate) was normal (data not shown). We concluded that the clofibrate treatment in line 7c males resulted in oligospermia from a partial block in spermatogenesis or spermiogenesis, and in functional sterility. Importantly, the pathology was very similar to that observed in CREM-null males (FIG. 3). In contrast, line 7c males on a regular diet were fertile—only after treatment with clofibrate did they develop oligospermia and become infertile.

We evaluated the residual sperm that was produced by the line 7c mice in more detail. We measured sperm motility with and without 2 mM clofibric acid and found no differences between WT and the mutants. We also have observed no differences in sperm morphology after H&E, “Christmas tree” (nuclear fast red and picroindigocarmine) or acridine orange stain. From all the above results, the combined lack of T1R3 and α-gustducin exerted its effect primarily at the level of developing spermatocytes in testis.

The figures show sections of a WT male (FIG. 3A, 3D) and a line7c male (3B, 3E) after 1 month on the clofibrate diet. PAS staining of testicular tubules (upper row) and epidydimi (lower row). Images of sections from CREM null males (2) illustrate a comparison (3C, 3F). Prominent and numerous PAS positive giant cells were present in testis and PAS-positive material with numerous immature cells and few spermatozoa in epididymis of Line7c.

The histological picture of the DKO animals showed testicular pathology very similar to that seen in the clofibrate-treated line 7c animals and CREM-nulls (FIG. 3). We observed a disruption of spermatogenesis or spermiogenesis with significant reduction of mature spermatocytes and the presence of Periodic-acid Shiff (PAS)—positive giant cells in testicular tubules, and the presence of immature cells with abundant PAS-positive material in epididymal tubules and cellular detritus but no moving sperm in epididymal tubules and vas deferens. Tubules in the epididymis showed PAS-positive material and cell debris that appeared more abundant than in the clofibrate-treated line 7c animals (FIG. 3G) Immunohistochemistry with protamine-2 antibody showed no staining of sperm heads in the double-null testis in contrast to WTs (data not shown).

If the chimeric mhT1R3 is blocked either genetically or pharmacologically, males developed oligospermia and functional sterility.

### Example 5

### Transmission Ratio Distortion (TRD)

From T1R3 knockout (KO) x α-gustducin (Gus) KO crosses, we uncovered transmission ratio distortion (TRD), where the Gus−/T1R3− haplotype was selectively not transmitted from males. The α-gustducin and T1R3 single nulls were maintained as homozygous lines. They reproduce normally and have normal life spans. We were unsuccessful in our attempt to produce T1R3/α-gustducin double-nulls. We genotyped embryos as early as embryonic day 13-15 and could not detect double homozygotes. Therefore, we concluded that the defect must be at an earlier stage.

Thus, we could not obtain and study double-null animals using conventional crosses, but required the development of Lines 7 and 7c as described above. In the Line 7 and Line 7c mice, the mhT1R3 transgene functionally replaced the endogenous receptor and allowed male reproduction to proceed.

To determine if either haploid sperm or eggs were defective, we set up a cross between Gus−/−/Gus+/+ and T1R3−/−/Gus+/− animals (FIG. 1). Surprisingly, we found that a haplotype that is both Gus− and T1R3− is not transmitted to progeny if it is produced from a male. The same haplotype carried by female germ cells is normally transmitted in Mendelian frequency. Because the gonadal environment of those compound homo- and heterozygotes can potentially influence development of the haploid cells, we set up a simpler and physiologically neutral cross: a double heterozygote crossed with a WT animal. In the double-heterozygote animal, all diploid cells should function normally because they express WT (+) alleles. As shown in FIG. 2, we again detected no transmission of the Gus−/T1R3− haplotype from males. Both experiments indicated that it is the haploid male germ cell that is defective when both genes were absent. It is unlikely that a hormonal regulation or a function of supporting Sertoli or Leydig cells is affected, because +/+ and +/− germ cells were produced and develop normally in these double heterozygotes and were passed on to progeny. It was only the −/− haploid cells that were not produced or were defective.

Thus, from T1R3 knockout (KO) x gustducin (Gus) KO crosses we uncovered transmission ratio distortion, where the Gus−/T1R3− haplotype was not transmitted selectively from males. It was transmitted in normal frequency from females, as shown in the tables below. (See Tables 1-4 below).

Table 1 shows the results of a transmission ratio distortion from T1R3 KO mice crossed with gustducin KO mice. Genotypes of progeny of crosses between male gustducin −\− and female gust+/−, T1R3−/− animals depend on the sex of parents. Compound hetero/homozygote is a female or a male.

TABLE 1
Male gust−/−, T1R3 + X Female gust+/−, T1R3−/−

| MATERNAL GAMETES | | |
|---|---|---|
| (paternal gametes are gust−/ | | ACTUAL |
| T1R3+ in all cases) | PROGENY | (EXPECTED) % |
| gust+, T1R3− | gust+/−, T1R3+/− | 4 (50%) |
| gust−, T1R3− | gust−/−, T1R3+/− | 6 (50%) |

Table 2 shows the results of a transmission ratio distortion from T1R3 KO mice crossed with gustducin KO mice. Genotypes of progeny of crosses between male gustducin+\−T1R3−/− and female gust−/−, animals depend on the sex of parents. Compound hetero/homozygote is a female or a male.

TABLE 2
Male gust+/−, T1R3−/− X Female gust−/−, T1R3+/+

| PATERNAL GAMETES | | |
|---|---|---|
| (maternal gametes are gust−/ | | ACTUAL |
| T1R3+ in all cases) | PROGENY | (EXPECTED) % |
| gust+, T1R3− | Gust+/−, T1R3+/− | 21 (50%) |
| gust−, T1R3− | Gust−/−, T1R3+/− | 0 (50%) |

Table 3 shows the results of a transmission ratio distortion showing that genotypes of progeny of crosses between double-heterozygote and wt animals depend on the sex of parents. Double heterozygote is a female (gust+/−, T1R3+/−).

TABLE 3
Wt Male gust + X Female gust+/−, T1R3+/−

| MATERNAL GAMETES | | |
|---|---|---|
| (paternal gametes are | | ACTUAL |
| gust+/T1R3+ in all cases) | PROGENY | (EXPECTED) % |
| gust+, T1R3+ | Gust+/+, T1R3+/+ | 3 (25%) |
| gust−, T1R3+ | Gust+/−, T1R3+/+ | 3 (25%) |
| gust+, T1R3− | Gust+/+, T1R3+/− | 3 (25%) |
| gust−, T1R3− | Gust+/−, T1R3+/− | 4 (25%) |

Table 4 shows the results of a transmission ratio distortion showing that genotypes of progeny of crosses between double-heterozygote and wt animals depend on the sex of parents. Double heterozygote is a male (gust+/−, T1R3+/−).

TABLE 4
Male gust+/−, T1R3+/− X Wt Female gust+/+

| PATERNAL GAMETES | | |
|---|---|---|
| (maternal gametes are | | ACTUAL |
| gust+/T1R3+ in all cases) | PROGENY | (EXPECTED) % |
| gust+, T1R3+ | Gust+/+, T1R3+/+ | 6 (25%) |
| gust−, T1R3+ | Gust+/−, T1R3+/+ | 6 (25%) |
| gust+, T1R3− | Gust+/+, T1R3+/− | 7 (25%) |
| gust−, T1R3− | Gust+/−, T1R3+/− | 0 (25%) |

This example demonstrated that T1R3− and gustducin− sperm did not contribute to the progeny. From this finding alone one cannot discern whether the T1R3−/gust− sperm is not produced in the animal or is defective.

Thus, in two independent genetic studies in mice we found no germ-line transmission when T1R3 and Gα-gustducin genes were missing in haploid male germ cells. In contrast, female haploid eggs missing both genes were fully functional, transmitting the genes (or lack thereof) to the progeny. A single deficiency in either T1R3 or Gα-gustducin has no effect on reproduction.

### Example 6

### Expression of T1R3, Gustducin and Crem in Testicular Tubules

As revealed in FIGS. 2A-2D, GFP fluorescence was examined in in germ cells in testicular tubules from wt mouse (2A) and T1R3-mhT1R3-IRES-GFP mouse (2B). Subsequent immunostaining was performed with anti-gustducin antibody (2C) and overlayed with GFP fluorescence (rD). In situ hybridization with T1R3 antisense probe on sections of tubules of WT males was performed and shown in (2E). Expression of CREM in testicular tubules from Ref 11 shown for comparison (2F). T1R3, gustducin and CREM were shown to be expressed in the developing spermatocytes in testes.

### Example 7

### PCR Analysis of Testis-Specific Transcripts

We isolated mRNA from testis of WT and the double-null DKO males. By qPCR we analyzed expression of genes that were controlled by CREM. We also analyzed levels of CREM transcript that originates from the alternative promoters Ψ1 and Ψ2 and is induced by cAMP. Our results indicated that expression of genes from developing spermatocytes, such as those encoding transitional proteins, and protamines was diminished, while expression of CREM Ψ1 and 2 and Egr 2 was elevated, compared with WT males (data not shown).

For male TRD to occur, the relevant gene products need to be compartmentalized, or to be unable to cross interspermatid bridges. Both α-gustducin and T1R3 were membrane-bound molecules that fulfill these criteria. We reasoned that although there is cooperation in function of both molecules, in testis they can also function independently and were able to partially complement each other's function. We hypothesize that this common function may be their ability to modulate cAMP levels. Indeed, both signaling molecules critically affect this second messenger (9-14).

This pointed us to downstream signaling molecules dependent on cAMP signaling that may be critically affected in male spermatogenesis or spermiogenesis. We noted that CREM (cAMP-responsive element modulator) is such a factor, indispensable for transcription of specific gene products necessary for development and maturation of spermatocytes. CREM is also expressed in the same germ cells as were T1R3 and α-gustducin.

We set up experiments from which we found that a pharmacological block of the mouse-human mhT1R3 chimeric receptor on the α-gustducin-null background results in severe reduction of viable spermatozoa and functional sterility. Moreover, from interbreeding of the mhT1R3 transgenics, we obtained two T1R3/α-gustducin double-null (DKO) males. Importantly, these males had no viable sperm. We propose the following genetic and pharmacological approach to test the hypothesis that absence or inhibition of T1R3 and Gα-gustducin affects function of the master switch CREM (cAMP-responsive element modulator), resulting in block of spermatogenesis or spermiogenesis, specifically, to determine which testicular cells were most affected by block of T1R3 and α-gustducin and how this impairs the function of CREM.

### Example 8

### Additional Studies

We pharmacologically blocked both T1R3 and Gα-gustducin pathways in mice expressing a humanized form of T1R3 susceptible to inhibition by lactisole and clofibrate and examine contraception. We used a pure pharmacological approach to block spermatogenesis or spermiogenesis in existing transgenic mouse lines Line 7 and Line 7c expressing a humanized form of T1R3. We used T1R3 inhibitors and indirectly blocked the signaling from gustducin by inhibiting phosphodiesterases (PDEs), which interact with α-gustducin and were expressed in testis.

In the examples above, we observed that Line 7 males during the pharmacological block of humanized mhT1R3 developed oligospermia and were functionally sterile. They produced about one-quarter the number of sperm produced by WT mice. Spermatozoa morphology was indistinguishable from that of WT males, and motility was not affected even when clofibric acid was added to the media (data not shown). We conclude that this sperm was produced by cells that escaped the pharmacological block.

To further characterize male reproductive functions after pharmacological block of the gustducin and T1R3 pathways, we tested the subhypotheses that this block in spermatogenesis or spermiogenesis and pharmacologically induced sterility will be reversible. Using Line 7c mice that express the humanized mhT1R3 transgene in the T1R3/α-gustducin double-null background, as well as existing T1R3-null (Gus+/+) lines, and mhT1R3+/T1R3-null (Gus+/+) mice, we characterized the effects of eliminating T1R3 function using phenoxy compounds such as fibrates. As described in the examples above, we determined that clofibric acid inhibition of the humanized mhT1R3 transgene significantly affected male fertility. The results demonstrated that the first step in male reproduction affected by the block of α-gustducin and T1R3 is at the level of spermatogenesis or spermiogenesis.

We used six mice per group to establish the levels of inhibitors necessary to elicit a complete block in spermatogenesis or spermiogenesis. We also determined if the pharmacological block is reversible. We blocked the humanized mhT1R3 in the α-gustducin-null background with clofibrate as we have done above. In Line 7, T1R3-null mice, we blocked the WT α-gustducin pathways pharmacologically. Gα-gustducin is very similar to retinal Gα-transducin, and its function is primarily to activate a phosphodiesterase (PDE). Although expression of several PDEs (PDE 1, 4, 8, and 11) has been reported in testis, which of these is critical in the cells expressing T1R3, α-gustducin, and CREM is not known. Therefore, we used theophylline as a nonspecific PDE inhibitor in the T1R3-null background to examine the effect of blocking α-gustducin's downstream signal.

We simultaneously inhibited both the humanized T1R3 and PDEs in the WT α-gustducin background to determine if we can block spermatogenesis or spermiogenesis solely by pharmacological means. We analyzed histological changes in testes and epididymi, and measured levels of key hormones even though CREM mutants showed no changes in their hormonal levels. In addition, we tested the reproductive capacity of double-null females that do or do not carry the transgene, although based on our initial data we expect no significant effect on female gametes.

For in vitro experiments we isolated tubular cell populations and analyzed their relative numbers and expression profile of genes that are hypothesized to be affected.

### Example 9

### Conditional Testes-Specific KO Mouse Line

To study the involvement of T1R3 in development, growth, and function of male reproductive cells, we selectively deleted T1R3 in germ cells in animals already lacking α-gustducin, using tissue-specific Cre expression.

We generated a tissue-conditional KO mouse line where LoxP sites flank T1R3. We cross this “foxed” line with a transgenic line expressing Cre from a testis-specific promoter to create a T1R3 deletion specifically in testis. With this conditional KO line, we selectively inactivated T1R3 in male germ cells and produced enough animals with the double null genotype for analysis. This mouse line differs from 7c in which mhT1R3 was expressed in all relevant tissues and Gα-gustducin was not expressed in all relevant tissues.

In the tissue-conditional transgenic mice, where Gα-gustducin is absent from all tissues and T1R3 is deleted conditionally in a tissue-specific manner, the male reproductive functioned as described below.

The T1R3 locus was successfully targeted in the past by two groups. A new animal model with a “floxed” T1R3 gene is currently being produced in our laboratory. To generate tissue-specific deletion, we consider that the haploid male germ cells defective in T1R3 and α-gustducin do not contribute to the progeny. Therefore, we cannot simply cross double heterozygotes (floxed T1R3, α-gustducin) expressing the testis-specific Cre because the Cre would readily produce nonfunctional haploid male germ cells. We generate such heterozygotes in females and then cross heterozygous females expressing Cre with heterozygous males without Cre. For example, we can cross a loxT1R3+/−, Gus−/− male with a loxT1R3+/−, Gus−/−, Pgk2-Cre (41) or Stra8-Cre (48) female. We expect one-fourth of the progeny to contain loxT1R3 on a Gus−/− background and express Cre. Half of these animals were females, which were used for further breeding. Cre is functional in testes of newborn males produced from this cross, creating the desired deletion of T1R3. The males were expected to be sterile. They were used for analysis.

Standard DNA manipulation techniques were used to produce the DNA construct. We opted to create targeted embryonic stem (ES) cells. Chimeric animals with the selected ES clone were bred first with WT animals, and after germline transmission the heterozygotes were crossed with a general “deletor line” such as Ella-Cre, expressing Cre from adenovirus promoter early in development in virtually all cells to verify the functionality of the construct. Next, animals were intercrossed with α-gustducin-KO animals to produce a double-homozygous line. For tissue-specific deletion, we use the Stra8-Cre animals (48) that express Cre in spermatogonia and spermatids. From the resulting animals, we establish a homozygous line. Standard breeding schemes were used to derive compound homo- and heterozygotes with and without Cre.

A number of existing mouse lines expressed Cre in various stages of male spermatogenesis or spermiogenesis (see 45-48). The most recent line is obtained from JAX (stock #008208; 48), where Cre is expressed from the Stra8 gene only in males beginning at postnatal day 3 in early-stage spermatogonia and is detected through pre-leptotene-stage spermatocytes. Alternatively, we used well-characterized Pgk2-Cre animals that express Cre also at this stage.

We already have T1R3-null mice in our lab, and they have normal life span and were fertile. We therefore expected no deleterious effects of the conditional T1R3 disruption. We can readily verify the integrity of the conditional T1R3 allele in a brief-access taste test, because T1R3-null animals were impaired in the detection of sweet and umami (amino acid) compounds, whereas the animals with a conditional allele should have normal taste ability.

First we introduced the floxed T1R3 into the α-gustducin-KO background (Gus−/−). This takes two generations. The F1 back-crossed to Gus−/− should yield floxed T1R3+/− and Gus−/−. These animals are crossed with both Cre lines. We then crossed the floxed T1R3/Gus−/− with Stra-8-CRE. From the F1 generation we selected animals that are floxed T1R3+/−/Gus+/−/Cre+/−. The expected frequency is 1:4. Then we intercrossed F1 into F2. From these progeny we selected several genotypes for final breeding: floxed T1R3+/−/Gus−/− with or without Cre, floxed T1R3+/+, Gus+/− with or without Cre. These should appear in 1:8 frequency. In 1:16 frequency we obtain double KOs with or without Cre. In the final breeding we used compound homo/heterozygous females carrying Cre and homo/heterozygous males (or double KOs) without Cre. The desired animals with T1R3 deleted in testis appear in the progeny of these parents, depending on the genotype on average in 1:8 frequency. The males were used in experiments and females for further breeding. We maintained the double-KO animals as a line (the floxed T1R3 is functional) and compound homo/heterozygous with Cre.

### Example 10

### Characterize Male Reproductive Functions

The mice from Example 9 were assessed for involvement of T1R3 and α-gustducin in development, growth, and function of male reproductive cells. The Cre expression from the Stra8 promoter inactivated the T1R3 gene very early in gonadal development: at postnatal day 3 in early-stage spermatogonia. Therefore, to determine the progression of testicular defect(s) from very young animals to adulthood, we compared the testicular anatomy and histology with WT animals. We examined H&E and PAS sections and use immunohistochemistry to visualize which cells remained in the tubules as well as RT-PCR to determine which genes were affected.

We used markers for Leydig and Sertoli cells such as 3beta-hydroxydehydrogenase, M2A, and anti-Mullerian hormone. A number of markers can distinguish various stages of spermatogenesis or spermiogenesis, such as Thy1 in spermatogonia, GCNA1 in spermatogonia and early spermatocytes, SOX-17 in round spermatids, GP90 in preleptotene to pachytene spermatocytes, Vps26a in pachytene spermatocytes, and Tnp1&2 in spermatids. We focused on the following gene products that were known to be affected by the absence of CREM and that we hypothesize were affected by the absence of T1R3 and α-gustducin: Tnp1 and 2, protamine 1 and 2, and calspermin.

We also looked for potential apoptotic cells with TUNEL analysis and caspase activation. We used ELISA to measure levels of testosterone, follicle-stimulating hormone, and leutinizing hormone that can be affected by abnormal spermatogenesis or spermiogenesis.

We compared WT animals with α-gustducin nulls with selective inactivation of T1R3. Information on other control genotypes, such as single-null KOs, is obtained from Example 1c. Anatomical, histological, and functional analysis of changes in testes and spermatozoa used the same methods as in Example 2.

### Example 11

### Analysis of Signaling Pathways

We analyzed the signaling pathways in WT and mutant animals from Examples 1-10 above to determine the role of cAMP and other signaling molecules and to analyze the molecular mechanisms by which α-gustducin and T1R3 affect downstream cAMP levels and CREM function and other downstream genes in spermatids.

We further assessed the intermediate signaling molecules and role of PDEs, and which genes were affected by lack of T1R3 and α-gustducin. We expect that spermatocytes in α-gustducin-null, T1R3-null, or α-gustducin/T1R3 double-null males have abnormal cAMP levels and that that expression of CREM and its downstream target genes is altered in α-gustducin/T1R3 double-null males.

In taste signaling, the T1R3 receptor interacts predominantly with the Gα-gustducin, but it also effectively couples with inhibitory Gαi subunits. In vitro studies have shown that purified T1R3, as well as T1R1 and T1R2 monomers, have constitutive activity. In cell culture, activation of the T1R2+T1R3 sweet receptor lowers cAMP levels. Conversely, T1R3 inhibition by lactisole results in a significant (3-fold) increase in cAMP levels. Because lactisole and other small-molecule inhibitors can increase cellular cAMP by actively decreasing the T1R2+T1R3 receptor's activity, these inhibitors were classified as inverse agonists. Thus, T1R3 is likely to have constitutive activity in vivo, which would result in lowering of cAMP levels.

A deletion of T1R3 would be predicted to result in a significant increase of cAMP. This is not a unique feature of T1R3; many GPCRs have constitutive activity, and many GPCR inhibitors were inverse agonists. α-gustducin is closely related to retinal transducins. Transducin's α-subunit activates retinal (type 6) phosphodiesterases (PDEs), and α-gustducin and α-transducin activate Ca2+/calmodulin (type 1) PDEs (unpublished data).

Interestingly, α-gustducin, or more likely the receptors that couple to it in taste cells, may also display constitutive activity in vivo. Lack of α-gustducin in KO mice produced a 4-fold increase of cAMP levels relative to wild-type (WT) mice and, in turn, abnormal activation of protein kinase A (PKA), which was sufficient to block physiological responses of taste cells. Restoring PKA function, even in the absence of α-gustducin, resulted in regained responsiveness of the cells.

In contrast to the taste system, in testis T1R receptors and α-gustducin may not operate in the same or single signaling pathway. In testis, in the absence of T1R3, α-gustducin could receive input from another receptor; while in absence of α-gustducin, T1R3 apparently couples to another G protein. When both molecules were blocked, a vital function is apparently lost. We hypothesize that cAMP is the common signaling molecule that is affected by the absence of T1R3 and α-gustducin. Severe disruption of cAMP signaling has a profoundly deleterious effect in spermatids because of the unique role of CREM. How CREM is affected by abnormal cAMP levels is not well understood. Production of specific CREM splice variants or use of CREM's alternative cAMP regulated promoters may be affected. Alternatively, PKA may be abnormally activated, resulting in phosphorylation of CREM protein. CREM in spermatids is normally not phosphorylated and binds to ACT (activator of CREM in testis) to initiate transcription of spermatid-specific genes. If phosphorylated, it would bind CBP (CREB binding protein) and start transcription of somatic genes. We determine the phosphorylation status of CREM in our mouse models.

We determined whether T1R3 and α-gustducin have a significant effect on cAMP levels in spermatocytes and spermatids and whether this affects CREM function. We have available individual KO models lacking either T1R3 or α-gustducin, and we block T1R3 function pharmacologically. We measured cAMP levels in these animals, including those produced in the examples above, in whole testis and in fractionated or FACS-sorted tubular cells. We measured the level of expression of CREM itself and changes in relative amounts of its variants. Importantly, we determined whether expression of genes controlled by CREM is affected. More than 50 genes (TISPs) were uniquely activated by CREM, and their expression is lost in CREM-null mutants. We also determined the phosphorylation level of the CREM protein. A prediction from our hypothesis is that treatment by agents that elevate cAMP levels (e.g., caffeine, forskolin) worsen the phenotype of the double-null mice; conversely, agents that lower cAMP may be beneficial.

A similar determination is made about modulation of intermediate signaling proteins such as PDE and PKA. We apply PDE inhibitors and novel PDE activators (e.g., Tradtrantip L, et al., 2009 Mol Pharmacol. 75(1):134-42). PDEs 1A, 1C, 4, 8A, and 11 were reportedly expressed in developing spermatocytes and spermatids. Moreover, PDEs 1A and 1C were directly activated by Gα-gustducin. Therefore, an interaction between Gα-gustducin and PDE 1 in testis is likely; consequently, PDE 1 inhibitors were tested first. Using other selective PKA inhibitors or activators, we further specify participants in the signaling pathway. By blocking both genes in vivo purely pharmacologically, we determined the effect on reproduction. We blocked mhT1R3 with clofibrate and gustducin pathway either with a non-specific PDE blocker or with a specific PDE1, 4, 8, or 11 blocker.

The methods employed in this example include isolation of tubular cells. Several protocols effectively separated various cell types from testicular tubules to high purity. We followed the sedimentation protocol in BSA gradient. We determined the cell identity by microscopic evaluation, RT-PCR analysis of stage-specific genes, and immunohistochemistry, using protocols such as described (e.g., Kotaja N, et al., 2004 Nat Methods. 1(3):249-54). For downstream molecules and signaling, we used α-gustducin-GFP, T1R3-GFP, and our recent humanized mhT1R3 transgenic animals that express GFP (Damek et al, cited above). We also have all the GFP transgenes on the null background of the parental gene (e.g. α-gustducin-GFP on the α-gustducin-null background). Therefore, we correlated GFP expression with the fractionated cells. Importantly, we isolated GFP positive cells by FACS sorting. We either separated the presumably less important GFP-negative cells after BSA fractionation, or further purified FACS-sorted cells by BSA sedimentation. We used freshly isolated cell populations for immediate measurements of cAMP and for gene expression studies and performed a short-term culture in the presence or absence of various inhibitors. Use of inhibitors in the background of single and double-null cells and measuring levels of cAMP point to the main actors in the affected signaling pathway(s). We used a nonspecific PDE inhibitor such as IBMX at 0.1-0.5 mM and PKA inhibitors (e.g., membrane-permeant H89 at 10 μM). Vinpocetine (IC50=10 μM) and rolipram (200-400 nM) were quite selective inhibitors of PDEs 1 and 4. Dipyridamole is useful for blocking PDE 8 with an IC50 of 4-9 μM, and PDE 11 can be blocked by the PDE 5 inhibitor tadalafil (IC50=5.5-75 nM). A large number of PDE resistant PKA activators (e.g., Sp-cAMP), including membrane permeant variants (http://www.biolog.de/no cache/products/eshop/product/A_{—}035/), was used to further dissect function of various players in this signaling cascade. cAMP and other biochemical parameters including hormone levels were measured.

To determine the expression of CREM and downstream genes, we use RT-PCR to determine expression of CREM, its transcriptional variants, Tnp, protamine, and other TISP genes essentially as described for Example 1. We used qPCR to determine relative expression levels of CREM-regulated genes in the pharmacological intervention experiments. By immunohistochemistry with specific antibody (e.g., sc-7978 detecting Ser133 in CREB and the corresponding and serine in CREM), we determined whether CREM is phosphorylated.

### Example 12

### The Upstream Roles of T1R1 and T1R2 Receptors

The pleasant tastes sweet and umami are mediated by a family of three T1R receptors that assemble into different receptors. The T1R2+T1R3 heteromer responds to sugars and sweeteners, whereas T1R1+T1R3 responds to glutamate and other amino acids described as savory or umami (Japanese for “delicious”). T1R3 alone, possibly as a homodimer, may serve as a low-affinity sweet receptor for carbohydrates. The T1R receptors were G-protein-coupled receptors (GPCRs) that in taste cells interact primarily with the G protein gustducin. T1R3, α-gustducin, and other signaling molecules present in gut enteroendocrine cells were implicated in nutrient sensing and the regulation of glucose metabolism through the release of intestinal hormones.

Both T1R3 and α-gustducin were expressed in testes and in spermatozoa. As evidenced in Examples 1-10, we found that T1R3 and α-gustducin were expressed in testicular tubular cells, predominantly in haploid spermatids (see FIG. 4, below).

T1R3 in taste cells pairs with T1R2 to form a sweet receptor and with T1R1 to form an amino acid receptor. Furthermore, T1R3 may function as a homodimer. Interestingly, T1R1 and T1R2 were also expressed in testis (data not shown), although their precise location is unknown. The upstream roles of T1R1 and T1R2 receptors that were also expressed in testis and were known to form functional heterodimers with T1R3 in taste cells were examined.

To determine the functional role of T1R1 and T1R2, we cross T1R1- and T1R2-null animals with α-gustducin nulls and analyze the progeny. We determine if the double homozygotes were born or whether there is a TRD as with T1R3. If double-null animals were born, we determine the status of spermatogenesis or spermiogenesis in testis sections and with H&E and PAS staining.

We determined whether T1R3 in testis signals in combination with T1R1 (forming an amino acid receptor) or with T1R2 (forming a sweet-sensing receptor) as it does in the taste system. We determined whether T1R1 or T1R2 receptors, which were known to couple with T1R3 in other areas, were involved in signaling with T1R3 in testis by examining whether T1R1- or T1R2-null alleles produce the same phenotype as T1R3-nulls.

These data help identify mechanisms and putative ligand/modifier(s) of taste receptors in testicular cells, which lead to the identification of agents that disrupt spermatogenesis or spermiogenesis and, conversely, agents that may improve male fertility. This greatly narrows possible natural ligand/modifier(s) of taste receptors in testis (amino acids T1R1) or sugars and sweeteners (T1R2), respectively).

By immunohistochemistry and in situ hybridization, we determined if T1R1 and/or T1R2 is expressed in the same testicular cells as T1R3. Importantly, we determined whether T1R1 or T1R2 have a functional role in testis. If T1R receptors function as heterodimers in testis as they do in taste cells, then blocking both α-gustducin and either T1R1 or T1R2 produces the same phenotype as blocking both α-gustducin and T1R3.

We expect to find, that the T1R1- or T1R2-null allele on a α-gustducin null background lead to transmission ratio distortion and result in blockade of spermatogenesis or spermiogenesis similar to that seen in CREM mutants and T1R3/α-gustducin double mutants.

Together, these data help identify mechanisms and putative ligand/modifier(s) of taste receptors in testicular cells, which lead to the identification of environmental or iatrogenic agents that disrupt spermatogenesis or spermiogenesis and, conversely, to the development of novel nonhormonal male contraceptives.

### Example 13

### Analysis of Desired Compositions that Modulate Fertility/Spermatogenesis or Spermiogenesis

We use the animal model expressing Gα-gustducin and the humanized T1R3 receptor as its sole T1R3 to develop a specific mix of compounds to block both gustducin and T1R3. We initially used fibrates together with a Gα-gustducin blocker that is effective against Gα-gustducin, such as suramin (Jones R. et al., Mol Hum Reprod 1996, 2: 597-605). A pharmacological high-throughput screening approach is then employed to identify compounds more specific in blocking gustducin. Similarly, even more potent T1R3 receptor blockers were developed.

In these examples, a novel transgenic mouse model presents a humanized form of the T1R3 receptor, which can be blocked with human-specific antagonists. This overcomes a limitation of existing rodent models in which their T1R3 does not respond like that of humans to inhibition by these compounds. The unexpected discovery obtained herein is that blocking two signaling molecules leads to a distinctive pathology such as male specific transmission ratio distortion (TRD) and male infertility. These examples show that rare males lacking both T1R3 and α-gustducin do not produce viable sperm and were sterile. In contrast, females missing both genes were fertile. Block of T1R3 and α-gustducin leads to transmission ratio distortion, oligospermia, and male sterility; and partial pharmacological block of T1R3 in the α-gustducin-null background resulted in oligospermia and effective male sterility in vivo. Histological changes in testis in the double-null animals and animals treated with clofibrate were very similar to those seen in CREM-null mice.

Total block of both T1R3 and α-gustducin lead to absence or significantly lower expression of genes controlled by CREM. The T1R3 or α-gustducin single-nulls have normal or slightly altered expression. Similarly, we predict that cAMP levels were significantly elevated only in the double-null animals and that the developing spermatids expressing both T1R3 and α-gustducin were particularly affected. As explained above, agents that further elevate cAMP to worsen the phenotype and agents that can lower cAMP or inhibit its downstream molecules improve the phenotype.

We have found that the dual absence or combined inhibition and absence, respectively, of the G-protein coupled receptor (GPCR) T1R3 and the G-protein subunit Gα-gustducin leads to transmission ratio distortion and male selective sterility. These two molecules independently of each other physiologically are theorized to downregulate cAMP and thus indirectly PKA. This downregulation makes cells' “tonus” low, so they were less sensitive to various stimulation, and at the appropriate time ready to respond. Therefore this kind of regulation reduces a signaling noise. If the cAMP is tonically high, the cells cannot respond or they respond inadequately.

Importantly, PKA signaling is not needed and is not used in spermatogenesis or spermiogenesis. In fact, there seem to be mechanisms preventing its activation (including T1R3 and gustducin), because PKA would stimulate expression of somatic genes through phosphorylation of CREB (in contrast to sperm-specific genes transcribed via unphosphorylated CREM) and that would interfere with normal sperm development.

All publications cited in this specification, including priority document U.S. Provisional patent application No. 61/623,244, filed Apr. 12, 2013, are incorporated herein by reference. While the invention has been described with reference to particular embodiments, it will be appreciated that modifications can be made without departing from the spirit of the invention. Such modifications are intended to fall within the scope of the appended claims.

1. A composition for modifying spermatogenesis or spermiogenesis or fertility in a mammalian subject comprising
(a) a ligand or modifier or reagent that modifies the activation, nucleic acid expression, protein expression, signaling or activity of one or more T1R3 receptors or an intermediate in the signaling pathways thereof in the subject's testis cells or germ cells;
(b) a ligand or modifier or reagent that modifies the activation, nucleic acid expression, protein expression, signaling or activity of G-α-gustducin, or an intermediate in the signaling pathways thereof in the subject's testis cells or germ cells,
wherein said modification alters a characteristic of spermatogenesis or spermiogenesis or of sperm, testes cells or germ cells produced by said subject.

2. A method of modifying spermatogenesis or spermiogenesis in a male mammalian subject comprising
(a) administering to said subject in need thereof a composition that modifies the activation, nucleic acid expression, protein expression, signaling or activity of one or more T1R3 receptors or an intermediate in the signaling pathways thereof in the subject's testis cells or germ cells;
(b) administering to said subject in need thereof a composition that modifies the activation, nucleic acid expression, protein expression, signaling or activity of G-α-gustducin, or an intermediate in the signaling pathways thereof in the subject's testis cells or germ cells, and
(c) optionally administering to said subject a composition that modifies the activation, nucleic acid expression, protein expression, signaling or activity of cyclic adenonsine monophosphate (cAMP) or a downstream intermediate in the signaling pathway of cAMP,
wherein said modification alters a characteristic of spermatogenesis or spermiogenesis or of sperm produced by said subject.

3. (canceled)

4. The method according to claim 2, wherein the intermediate in the G-α-gustducin signaling pathway is a cyclic nucleotide phosphodiesterase (PDE).

5. The method according to claim 2, wherein the downstream intermediate in the cAMP signaling pathway is protein kinase A (PKA).

6. (canceled)

7. The method according to claim 2,
wherein the contacting occurs in vivo and comprises administering the compositions to a male subject or wherein the administering comprises contacting the male germ cells ex vivo or treating a sample of a mammalian subject's sperm ex vivo with a sufficient amount of the compositions (a)-(c) or
wherein the administering comprises contacting the male germ cells ex vivo or treating a sample of a mammalian subject's sperm ex vivo with a sufficient amount of the compositions (a)-(c).

8-10. (canceled)

11. The method according to claim 2, wherein composition (c) is a PDE inhibitor, wherein administration thereof increases, up regulates or activates the activation, expression, signaling or activity of cAMP.

12. The method according to claim 2, wherein composition (a) comprises lactisole, a phenoxy herbicides, 2,4,DP, 2,4,-5TPP, MCPP, 2,4,D, clofibric acid, gemfibrozil, bezafibric acid, gymnemic acid, an inhibitor of gustducin, an inhibitor of the G a subunit of gustducin, an inhibitor of protein lipase C (PLC) beta or an inhibitor of protein kinase A.

13. The method according to claim 7, wherein the composition (b) comprises one or more of an inhibitor of gustducin, an inhibitor of the G α subunit of gustducin, an inhibitor of protein lipase C (PLC) beta, an inhibitor of PKA, or an inhibitor of PDE.

14-15. (canceled)

16. The method according to claim 2, wherein composition (c) comprises a PDE activator or a PKA inhibitor, which decreases, down regulates, inhibits or blocks the activation, expression, signaling or activity of cAMP.

17. The method according to claim 16, wherein composition (a) comprises a sweetener that interacts specifically with the T1R3 subunit, sodium cyclamate, dihydrochalcon-related sweeteners, a peptide or protein or a D-amino acid acting on T1R1/T1R3 dimers, a sugar, an artificial sweetener, sucrose, maltose, fructose, a sweet alcohol, sorbitol, xylitol, erythritol, aspartame, sucralose, saccharin or monosodium glutamate and amino acids.

18. The method according to claim 2, wherein the composition (b) comprises one or more of an activator of gustducin, an activator of the G a subunit of gustducin, an activator of protein lipase C (PLC) beta, an activator of PKA, or an activator of PDE.

19-23. (canceled)

24. The method according to claim 2, wherein the contacting occurs during collection, treatment, storage, transportation, or administration of sperm as part of an in vitro fertilization (IVF) procedure.

25-26. (canceled)

27. A method for screening a test molecule for its effect on fertility comprising:
(a) contacting a mammalian sperm cell, testis cell or cell line expressing a T1R3 receptor and G-α-gustducin ex vivo with a test molecule;
(b) assaying the contacted cells or cell lines for a change in a physical or functional characteristic of the contacted cell or cell line in comparison with a reference cell or cell line contacted with a control molecule;
wherein a change in the physical or functional characteristic of the test molecule contacted cells or cell lines vs. the reference indicates a modifying effect of the test molecule on the quality, quantity or viability of the sperm.

28. The method according to claim 27, wherein the assaying step comprises:
assaying the contacted cell or cell line for a change indicative of T1R3 receptor activation, expression, signaling or activity and G-α-gustducin activation, expression, signaling or activity in comparison with a reference cell or cell line contacted with a control molecule;
wherein a change in a characteristic or activity of the test cell or cell line in comparison to a reference indicates a modifying effect if the test molecule on the quality, quantity or viability of sperm.

29-30. (canceled)

31. The composition according to claim 1 for decreasing fertility in a mammalian subject comprising
(a) a ligand or modifier or reagent that decreases, inhibits, or down-regulates the activation, expression, or activity of one or more T1R3 receptors or an intermediate in the signaling pathways thereof in the subject's testis cells, germ cells or sperm; and
(b) a ligand or modifier or reagent that decreases, inhibits, or down-regulates the activation, expression, or activity of G-α-gustducin, or an intermediate in the signaling pathways thereof in the subject's testis cells, germ cells or sperm.

32. The composition according to claim 31, further comprising a reagent that increases, activates, or up-regulates the activation, expression, signaling, or activity of cAMP.

33. The composition according to claim 1, which is
(a) a topical cream, ointment, or transdermal patch for topical delivery of the ligand or modifier or reagent,
(b) an implant for timed release in vivo of the composition,
(c) present in or coated onto a condom or other contraceptive implant or device,
(d) designed for intravaginal contraceptive administration to a female subject, wherein said ligand or modifier is present in an amount sufficient to decrease the quality and/or quantity of sperm with which it comes in contact; or
(e) comprises other known contraceptive agents.

34-36. (canceled)

37. The composition according to claim 1, for increasing fertility in a mammalian subject comprising
(a) a ligand or modifier or reagent that increases, activates, or up-regulates the activation, expression, or activity of one or more T1R3 receptors or an intermediate in the signaling pathways thereof in the subject's testis cells, germ cells or sperm; and
(b) a ligand or modifier or reagent that increases, activates, or up-regulates the activation, expression, or activity of G-α-gustducin, or an intermediate in the signaling pathways thereof in the subject's testis cells, germ cells or sperm.

38. The composition according to claim 37, further comprising a reagent that decreases, inhibits, or down-regulates the activation, expression, signaling or activity of cAMP.

39. The composition according to claim 37, which is a composition for collecting, storing or transporting or administering sperm for insemination or IVF procedures.

40. (canceled)
